(19) United States
(12) Patent Application Publication
(10) Pub. No.: US 2007/0269420 A1
(43) Pub. Date: Nov. 22, 2007

(54) Compounds, Compositions and Methods for Treatment and Prophylaxis of Hepatitis C Viral Infections and Associated Diseases

(76) Inventors: Srinivas K. Chunduru, West Chester, PA (US); Christopher A. Benatatos, Downingtown, PA (US); Theodore J. Nitz, Pottstown, PA (US); Thomas R. Bailey, Phoenixville, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/579,813

(22) PCT Filed: Nov. 24, 2004
(86) PCT No.: PCT/US04/39533

§ 371 Date: Apr. 12, 2007

Related U.S. Application Data
(60) Provisional application No. 60/525,042, filed on Nov. 24, 2003.
(60) Provisional application No. 60/526,383, filed on Dec. 2, 2003.
(60) Provisional application No. 60/526,198, filed on Dec. 2, 2003.
(60) Provisional application No. 60/526,196, filed on Dec. 2, 2003.
(60) Provisional application No. 60/526,199, filed on Dec. 2, 2003.
(60) Provisional application No. 60/526,247, filed on Dec. 2, 2003.
(60) Provisional application No. 60/526,248, filed on Dec. 2, 2003.
(60) Provisional application No. 60/526,220, filed on Dec. 2, 2003.

(51) Int. Cl. A61K 31/164; A61K 31/135; A61K 31/165; A61K 31/38; A61K 31/415; A61K 31/505; A61K 31/52; A61K 31/53; A61K 31/7105; A61K 31/711; A61K 39/395; A61P 31/12; C12N 5/00; C12Q 1/02; C12Q 1/68; C12Q 1/70; G01N 33/00; G01N 33/53

(52) U.S. Cl. 424/130.1; 435/29; 435/375; 435/5; 435/6; 435/7.1; 436/86; 514/243; 514/259.3; 514/263.1; 514/275; 514/406; 514/44; 514/444; 514/448; 514/617; 514/647; 514/789

(57) ABSTRACT

The present invention is directed to compounds, compositions and methods for the treatment or prophylaxis of viral infections and diseases associated therewith, particularly those viral infections and associated diseases caused by the hepatitis C virus. Also within the scope of this invention are assay methods for identifying agents that modulate NS4B activity, as well as kits for performing such methods.

### CROSS-REFERENCE TO RELATED APPLICATIONS

This application claims the benefit of U.S. Provisional Application Nos. 60/525,042, filed Nov. 24, 2003, 60/526,383, filed Dec. 2, 2003, 60/526,220 filed Dec. 2, 2003, 60/526,198, filed Dec. 2, 2003, 60/526,196, filed Dec. 2, 2003, 60/526,199, filed Dec. 2, 2003, 60/526,247, filed Dec. 2, 2003, and 60/526,248, filed Dec. 2, 2003.

The entire disclosure of each of the aforementioned provisional patent applications is incorporated by reference herein.

### FIELD OF THE INVENTION

The present invention relates to compounds, compositions and methods for the treatment or prophylaxis of viral infections and diseases associated therewith, particularly those viral infections and associated diseases caused by the hepatitis C virus.

### BACKGROUND OF THE INVENTION

Hepatitis C is a common infection that can lead to chronic hepatitis, cirrhosis, liver failure, and hepatocellular carcinoma (HCC). Infection with the hepatitis C virus (HCV) leads to chronic hepatitis in at least 85% of cases. It is the leading reason for liver transplantation, and is responsible for at least 10,000 deaths annually in the United States (Hepatology, 1997, 26 (Suppl. 1), 2S-10S).

The duration from the onset of acute hepatitis until the time of diagnosis of cirrhosis of the liver and of HCC is about 20 and 30 years, respectively. The acute phase lasts from the onset of disease until 2-3 years thereafter, and the silent phase which follows lasts for 10-15 years. Since so little is known about the biology of HCV, it is presently unclear how this RNA virus establishes a persistent infection.

The hepatitis C virus is a member of the Flaviviridae family. The genome of HCV is positive strand, single stranded linear RNA (Hepatology, 1997, 26 (Suppl. 1), 11S-14S). HCV displays extensive genetic heterogeneity; at least six genotypes and more than 50 subtypes have been identified.

Following infection by HCV, the viral RNA is translated into a polyprotein. This approximately 3,000 residue polyprotein is subsequently cleaved into individual proteins by host peptidases, as well as virally encoded proteases. The HCV genome encodes structural proteins (required for virus assembly) and nonstructural proteins (required for replication). Some of the nonstructural proteins include: NS2, NS3, NS4A, NS4B, NS5A, and NS5B (J. General Virology, 2000, 81, 1631-1648).

The non-structural protein 4B (NS4B) of HCV is a small hydrophobic protein consisting of 6 transmembrane domains. Although this protein has no known enzymatic function, it is essential to viral replication. Structurally, HCV NS4B resembles the G-protein coupled receptor (GPCR) family of proteins. GPCRs are a superfamily of proteins responsible for mediating transmembrane signal transduction through GTP binding proteins or G proteins. The HCV NS4B protein may exert an agonist or antagonist effect on one or more innate cellular pathways in order to optimize the cellular environment for viral replication. While not being bound by any particular theory, these pathways may include the interferon (IFN α, β, γ) pathways, which are transduced via JAK/STAT family of transcriptional activators ultimately leading to the activation of ISGF (interferon stimulated gene family) and/or interferon response elements (IR). Another potential target pathway modulated by NS4B is the Endoplasmic reticulum (ER) Stress Response. In this case NS4B may act to block the activation of this pathway. The ER stress response is a cellular response to ER stress (i.e. accumulation of misfolded proteins, expression of viral proteins, etc.) where Flaviviral replication is known to take place. A family of cellular proteases known as Caspases modulates this pathway. Caspase 12 has recently been identified as a specific modulator of ER stress, signaling to caspase 9 and ultimately to caspase 3, which promotes apoptosis. Other target pathways potentially modulated by NS4B include the Protein Kinase R (PKR), the RNase L pathway, the 2′-5′ oligoadenylate pathway (OAS) and the Nuclear factor of transcription kappa B (NF-κB) pathway.

Interferon and interferon in combination with ribavirin are used in the U.S. for hepatitis due to HCV. These treatments are associated with improved serum enzyme response in some patients. The remainder are non-responsive to treatment. For responders, a sustained clinical improvement is seen in only a small percentage of patients; the majority of patients relapse upon cessation of treatment. Thus, the effectiveness of therapy for chronic hepatitis C is variable and its cure rate remains low. Moreover, therapy is often associated with considerable side effects.

Vaccines under development for HCV generally consist of recombinant versions of the putative viral structural proteins (C, E1, E2), or genes encoding these. It is believed that virus neutralizing antibodies do exist, can be elicited, and may be able to inhibit or prevent HCV infection. However, to date, no vaccine has been demonstrated to be safe and effective for HCV. Indeed, given the inherent genetic diversity of HCV, with virus isolates exhibiting immunologically distinct envelope proteins that are not neutralized by pre-existing antibodies, vaccine development will be a formidable task.

New therapies and preventatives are clearly needed for infections and diseases caused by the hepatitis C virus.

### SUMMARY OF THE INVENTION

It has been discovered in accordance with this invention that NS4B is a signal transducing molecule which modulates immune regulation and inhibits or prevents apoptosis in virally infected cells, thereby contributing to viral persistence. The compounds, compositions and methods of this invention are effective for the treatment and prophylaxis of HCV by inhibiting NS4B functions, and thereby interfering with the ability of the virus to replicate its RNA genome and produce progeny viruses.

According to one aspect of this invention, there is provided a compound or compounds which have NS4B signal transducing inhibitory activity, and which are effective to induce apoptosis in NS4B expressing cells that exhibit reduced apoptosis in the absence of such compound(s), the activity being determined by an NS4B binding assay method comprising exposing the compound to NS4B and determining the NS4B binding constant for the compound.

According to another aspect of this invention, there is provided a compound or compounds which have NS4B signal transducing inhibitory activity, and which are effective to induce apoptosis in NS4B expressing cells that exhibit reduced apoptosis in the absence of such compound, the NS4B signal transducing activity being determined by an assay method comprising contacting cells comprising an HCV replicon with the compound(s) and analyzing the cells for apoptosis, the compound(s) of the invention being the one(s) inhibiting NS4B signal transduction, and thereby stimulating apoptosis, relative to cells not contacted with the compound(s).

Among the compounds of the invention are those selected from the group having the following general formulas: 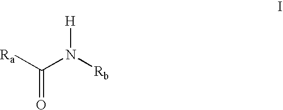
wherein:

Rₐ represents a radical selected from the group consisting of cycloalkyl, a heterocyclic radical, a substituted or unsubstituted aryl group, and a substituted or unsubstituted heteroaryl group;

R_{b} represents a radical selected from the group consisting of a substituted or unsubstituted aryl group and a substituted or unsubstituted heteroaryl group;

said aryl group substituents and said heteroaryl group substituents being one or more radical(s) independently selected from the group consisting of alkyl, alkoxy, halogen, phenylamido, a heterocyclic radical, and a substituted or unsubstituted heterocyclosulfonyl;

said heterocyclosulfonyl substituents being one or more radical(s) independently selected from the group consisting of a heteroaryl group;

and pharmaceutical salts thereof;

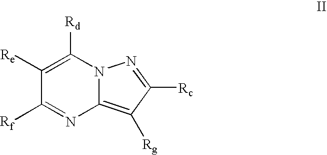

wherein R_{c} represents a radical selected from the group consisting of a substituted or unsubstituted aryl group and —C(═O)NH—Rₕ;

R_{d} represents a radical selected from the group consisting of hydroxy and polyhaloalkyl;

Rₑ represents a radical selected from the group consisting of hydrogen, alkyl, alkenyl, and arylalkyl;

R_{f} represents a radical selected from the group consisting of alkyl, phenyl and a heteroaryl group;

R_{g} represents a radical selected from the group consisting of hydrogen and alkyl;

Rₕ represents a radical selected from the group consisting of cycloalkyl, arylalkyl, and heteroarylalkyl;

said aryl group substituents being one or more radical(s) independently selected from the group consisting of alkyl, alkoxy, and halogen;

and pharmaceutical salts thereof;

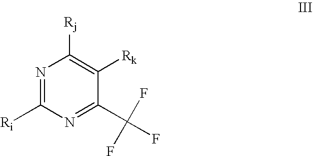

wherein Rᵢ represents a radical selected from the group consisting of amino, hydroxy, and a substituted or unsubstituted heterocyclic radical;

Rⱼ represents a radical selected from the group consisting of a substituted or unsubstituted aryl;

Rₖ represents a radical selected from the group consisting of hydrogen, alkyl, a substituted or unsubstituted aryl, and a substituted or unsubstituted heteroaryl;

said aryl group substituents, said heterocyclic radical substituents, and said heteroaryl group substituents being one or more radical(s) independently selected from the group consisting of alkyl, alkoxy, and halogen;

and pharmaceutical salts thereof;

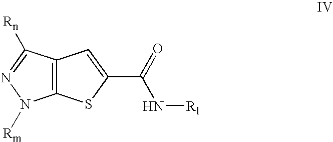

wherein Rₗ and Rₘ represent radicals that are independently selected from the group consisting of a substituted or unsubstituted aryl group and a substituted or unsubstituted heteroaryl group;

and Rₙ represents a radical selected from the group consisting of an alkyl group;

said aryl group substituents and said heteroaryl group substituents being one or more radical(s) independently selected from the group consisting of alkyl, alkoxy, halogen;

and pharmaceutical salts thereof;

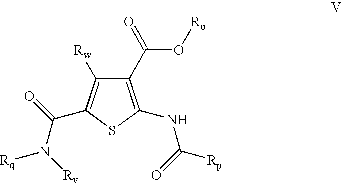

wherein Rₒ represents a radical selected from the group consisting of an alkyl group;

Rₚ represents a radical selected from the group consisting of alkyl, aralkyl, heteroaralkyl, a bicyclic heterocycle, a substituted or unsubstituted aryl group, a substituted or unsubstituted heteroaryl group, and a substituted or unsubstituted aryloxyalkyl group;

R_{q} represents a radical selected from the group consisting of alkyl, cycloalkyl, and a substituted or unsubstituted aryl group;

Rᵥ represents a radical selected from the group consisting of hydrogen and alkyl;

R_{w} represents a radical selected from the group consisting of an alkyl group;

said aryl group substituents, said heteroaryl group substituents, and said aryloxyalkyl group substituents being one or more radical(s) independently selected from the group consisting of alkyl, alkoxy, and halogen;

and pharmaceutical salts thereof;

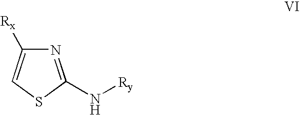

wherein Rₓ represents a radical selected from the group consisting of a substituted or unsubstituted aryl group and a substituted or unsubstituted heteroaryl group;

R_{y} is selected from the group consisting of a substituted or unsubstituted aryl group and a substituted or unsubstituted heteroaryl group;

said aryl group substituents and said heteroaryl group substituents being one or more radical(s) independently selected from the group consisting of alkyl, alkoxy, halogen, carboxyl, amino, amido, alkylcarbonyl, alkoxycarbonyl, and —SO₂—(NH)—R_{z}; and

R_{z} represents a radical selected from the group consisting of hydrogen and a heteroaryl group;

and pharmaceutical salts thereof; 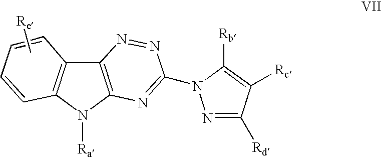
wherein:

R_{a′} represents a radical selected from the group consisting of hydrogen, alkyl, hydroxyalkyl, alkoxyalkyl, alkylthioalkyl, and dialkylaminoalkyl;

R_{b′} represents a radical selected from the group consisting of hydrogen, alkyl, alkoxy, hydroxyalkyl, aryl, and heteroaryl;

R_{c′} represents a radical selected from the group consisting of hydrogen, alkyl, alkoxy, hydroxyalkyl, aryl, and heteroaryl;

R_{d′} represents a radical selected from the group consisting of hydrogen, alkyl, alkoxy, hydroxyalkyl, aryl, and heteroaryl; and

R_{e′} represents a radical selected from the group consisting of alkyl, alkoxy, halogen, monoalkylamino, dialkylamino, and heteroaryl;

and pharmaceutical salts thereof; and 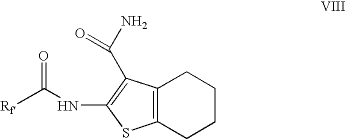
wherein

R_{f′} is selected from the group consisting of alkoxy, benzyl, and a substituted or unsubstituted phenyl;

said phenyl group substituents being one or more radical(s) independently selected from the group consisting of alkyl, alkoxy, and halogen;

and pharmaceutical salts thereof.

The invention also provides pharmaceutical compositions containing the antiviral compounds of Formulas I-VIII, above, and methods of using such compounds for treating and preventing infections caused by hepatitis C virus, as well as diseases associated with such infections in a living host.

The compounds of formula VII, above, modulate NS4-B associated apoptosis, and are effective for inducing apoptosis of HCV-infected cells in a patient, without producing toxemia in the patient. These compounds are beneficially administered for the treatment of HCV infection during the acute or silent phase of the infection, at a time prior to the patient's requiring hospitalization.

The compounds of formula VIII exhibit NS4B-associated signal transduction modulating activity, and are effective to inhibit HCV replication.

According to another aspect of the invention, assay methods are provided for the identification of agents that interact with NS4B, and, in particular those that modulate HCV NS4B associated apoptosis. Such methods include high throughput screening procedures that allow assessment of large numbers of agents. One such method for identifying compounds that modulate NS4B-associated apoptotic inhibitory activity comprises providing a host cell wherein NS4B is expressed; contacting the host cell with a test compound suspected of modulating NS4B associated apoptotic activity; and assessing such modulation as a function of alterations in apoptosis levels in the presence of the test compound. There are a variety of assay methodologies well known to the trained artisan that allow the efficient screening of large numbers of samples [see, for example, Cole, J L, in Meth. Enzymology 275:310-328 (1996)], and may utilize any number of activity detection and measurement technologies including, but not limited to, radiometric, calorimetric, fluorogenic, or chemiluminescent, any one of which may be suitable in the case of the HCV NS4B apoptosis modulating activity. The agents identified by use of the HCV NS4B assay method may be either antagonistic or agonistic in their affect on the NS4B associated apoptosis. These agents may include molecules of any number of classes including but not limited to small molecules, polymers, peptides, polypeptides, immunoglobulins or fragments thereof, oligonucleotides, antisense molecules, peptide-nucleic acid conjugates, ribozymes, polynucleotides and the like. It is specifically contemplated that both antagonistic and agonistic molecules identified by practice of the invention have broad and multiple utilities. Such utilities for antagonists of HCV NS4B activity include, but are not limited to, uses for the inhibition of HCV replication in humans, in other living hosts and in in vitro systems such as cell, tissue and organ cultures. Agonists of HCV NS4B activity identified by practice of the invention will also have multiple utilities, both in living hosts and in in vitro systems. For example, such agents will be useful in the development of animal models of HCV infection, replication or disease and for the propagation of HCV in a living host or in cell, tissue or organ culture systems.

In accordance with a further aspect of this invention, there is provided a method for identifying compounds having binding affinity for NS4B comprising: providing NS4B protein which is naturally fluorescent; contacting the NS4B protein with a test compound suspected of having binding affinity for such NS4B; and determining the fluorescence level of the NS4B protein in the presence and absence of such test compound, any agent which diminishes the natural fluorescence of NS4B being one that has binding affinity for NS4B.

Methods for assessing the signal transducing activities of NS4B are also provided in accordance with this invention. Representative methods include detection of HCV replication as a function of production of viral proteins in the presence and absence of candidate compounds. Down regulation of interferon stimulated gene expression can be assessed using host cells comprising reporter genes operably linked to promoters comprising interferon response elements and HCV replicons. Such host cells are contacted with candidate compounds and the ability of the compound to modulate interferon stimulated gene expression, either inhibition or stimulation, is assessed as a function of reporter gene expression levels. Further, an exemplary method for assessing NF-κB signaling entails providing host cells comprising reporter genes operably linked to promoters comprising NF-κB binding sites and HCV replicons. Such host cells are contacted by the candidate compounds and the ability to activate NF-κB signaling is assessed as a function of reporter gene expression levels. Other signaling pathways that may be assessed in this way include the endoplasmic reticulum stress response pathway, the RNase L pathway and the 2′5′ oligoadenylate pathway. An HCV protein binding assay may also be used for further characterization of the anti-viral activity of the candidate compounds.

The high throughput assay methods of this invention may also be used to study the influence of NS4B in the cellular proliferation associated with hepatic neoplasms induced by HCV infection.

The compounds identified by the assay methods described herein may also be used in a method of treating HCV infection in a patient in need of such treatment.

According to still another aspect of the invention, kits are provided to facilitate the use of the compositions and assay methods disclosed herein. Representative kits would include HCV NS4B nucleic acids and polypeptides of the invention, variants thereof, alone or in association with suitable vectors. Also included would be pertinent assay protocols for use of the kits and the necessary reagents to carry out the protocols. Examples of suitable means for determining apoptosis include, without limitation, measurement of DNA integrity, TUNEL assay and trypan blue exclusion assay. The reagents of a kit may vary depending on the intended application. Such reagents may include, but are not limited to buffers, solvents, media and solutions, substrates and cofactors, vectors and host cells, and detection or reporter reagents. Other accessories may also be included such as vials, vessels and reaction chambers.

Also in accordance with the present invention, there is provided a method of distinguishing NS4B biological activity from cellular chemical cytotoxicity exhibited by a test compound, the method comprising: measuring the apparent cytotoxicity of a test compound in a host cell system, measuring chemical cytotoxicity produced by the test compound in the host cell system containing NS4B protein, comparing the results of such measurements, and identifying the apparent cytotoxicity as corresponding to NS4B biological activity or chemical cytotoxicity.

The present invention further involves the discovery of the role of NS4B in modulating apoptosis, i.e., programmed cell death. Accordingly, another aspect of this invention is a method for inhibiting apoptosis in a target cell by administering to, or contacting the cell with an effective amount of NS4B.

In a further aspect of the invention, HCV NS4B proteins may be modified by particular changes in nucleotide and amino acid sequence that result in NS4B proteins with altered functionality. Such changes may be subtle and represent conservative substitutions such as in the case of nucleotide sequences, changes in the codon sequence that do or do not alter the encoded amino acid, or for amino acid sequences, changes that result in conservative residue substitutions, additions or deletions.

### BRIEF DESCRIPTION OF DRAWINGS

FIG. 1 shows the wild type nucleic acid sequence of HCV NS4B (SEQ ID NO: 1) and the protein sequence encoded thereby (SEQ ID NO: 2).

FIG. 2 shows a variant of the NS4B nucleic acid sequence (SEQ ID NO: 3) and the protein sequence encoded thereby (SEQ ID NO: 4).

FIGS. 3A-3G show a table of viruses containing NS4B-like proteins and the GenBank Accession numbers therefor.

### DETAILED DESCRIPTION OF THE INVENTION

The compounds of Formulas I-VIII above, their isomers and pharmaceutically acceptable salts exhibit antiviral activity. The compounds of the invention are particularly effective against hepatitis C virus and are useful in the prophylaxis and/or treatment of infections and diseases associated with this virus in living hosts.

In vitro studies (cell-based) have been performed which demonstrate the usefulness of compounds described herein as antiviral agents. For example, antiviral activity of representative compounds was evaluated in a human liver-derived cell line containing an HCV replicon.

The following definitions are provided to aid in understanding the various aspects of the present invention.

As used herein, the term “compounds of the invention” means, collectively, the compounds of Formulas I-VIII, pharmaceutically acceptable salts thereof, and mixtures thereof, as well as compounds identified by the assays described herein. Certain compounds of the invention are identified herein by their chemical structure and/or chemical name. Where a compound is referred to by both a chemical structure and a chemical name, and that chemical structure and chemical name conflict, the chemical structure is determinative of the compound's identity.

The term “alkyl” as used herein refers to straight or branched chain aliphatic hydrocarbon radicals of up to 10 carbon atoms, preferably up to 6 carbon atoms and more preferably 1 to 4 carbon atoms. Similarly, the term “alkyl” or any variation thereof, used in combination form to name substituents, such as alkoxy (—O-alkyl), aralkyl (-alkyl-aryl), heteroaralkyl (-alkyl-heteroaryl), alkoxyalkyl (-alkyl-O-alkyl), hydroxyalkyl (-alkyl-OH), monoalkylamino (—NH-alkyl), dialkylamino (—N-(alkyl)-(alkyl)), dialkylaminoalkyl (-alkyl-N-(alkyl)-(alkyl)), alkylthio (—S-alkyl), alkylthioalkyl (-alkyl-S-alkyl), or the like also refers to straight or branched chain aliphatic hydrocarbon radicals of up to 10 carbon atoms, preferably 1 to 6 carbon atoms, and more preferably of 1 to 4 carbon atoms.

The term “alkenyl” as used herein refers to straight or branched chain aliphatic hydrocarbon radicals of 2 to 7 carbon atoms containing at least one double bond. Such alkenyl moieties may exist in the E or Z configurations; the compounds of this invention include both configurations.

The term “phenyl” as used herein refers to a 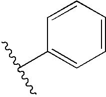
group. A “substituted phenyl” refers to a phenyl group that is substituted with the indicated substituents.

As used herein, the term “aryl”, refers to an aromatic carbocyclic group, having 6 to 10 carbon atoms including, without limitation, phenyl and napthyl. “Aryl” is sometimes used in combination form, e.g. “aralkyl” to refer to an aryl-substituted “alkyl” radical the latter being defined as above. A specific example of an aralkyl substituted is benzyl.

As used herein, the term “cycloalkyl” refers to non-aromatic carbocylic groups, having 3 to 7 carbon atoms, as for example cyclopropyl, cyclobutyl, cyclopentyl, cyclohexyl and the like.

The term “heterocyclic,” as used herein, refers to a or non-aromatic cyclic group having in the ring at least one carbon atom and at least one nitrogen atom and zero to four additional heteroatoms independently selected from oxygen, nitrogen or sulfur atoms. The point of attachment of a heterocyclic radicals can either be radical is through a carbon atom or a heteroatomnitrogen atom on the heterocyclic radical or a nitrogen atom on the heterocyclic radical. Heterocyclic radicals preferably have 3 to 10 members, and more preferably 4, 5, or 6 members in the ring. Examples of heterocyclic radicals include piperazinyl, piperidinyl, morpholinyl, pyrrolidinyl, imidazolidinyl, pyrazolyl and the like.

The term “amido,” as used herein, refers to a radical or substituent of the formula —NR″C(═O)R′″, wherein R″ and R′″ independently represent hydrogen, alkyl, or cycloalkyl. Similarly, the term “phenylamido,” as used herein, refers to a radical or substituent of the formula —NR″C(═O)phenyl, wherein R″ and phenyl are as previously defined.

The term “heterocyclosulfonyl,” as used herein, refers to a radical or substituent of the formula —SO₂—HET, wherein HET is a heterocyclic group as defined above. Preferred heterocyclosulfonyl groups include piperidinylsulfonyl and morpholinylsulfonyl pyrazolyl.

The term “heteroaryl,” as used herein, refers to a 5- or 6-membered aromatic cyclic group having at least one carbon atom and one or more oxygen, nitrogen or sulfur atoms in the ring, as for example furyl, thienyl, pyridyl, pyrrolyl, oxazolyl, thiazolyl, imidazolyl, pyrazolyl, isoxazolyl, isothiazolyl, 1,2,3-oxadiazolyl, 1,2,4-oxadiazolyl, 1,2,3-triazolyl, 1,2,4-triazolyl, 1-3-oxathiolanly, thiadiazolyl, tetrazolyl, triazolyl and the like. “Heteroaryl” is sometimes used in combination form, e.g. “heteroaralkyl” to refer to a heteroaryl-substituted “alkyl” radical, the altter being defined as above.

The term “aryloxy,” as used herein, refers to a radical or substituent of the formula —O-aryl, wherein aryl is as defined above. Likewise, the term “aryloxyalkyl,” as used herein, refers to an alkyl group, as defined above, further substituted with an aryloxy group.

The term “bicyclic heterocycle,” as used herein, refers to a bicyclic ring system where a phenyl ring is fused to a 5 or 6-membered saturated or partially saturated heteroaryl group, as defined above, containing 1 to 4 heteroatoms selected from the group consisting of S, N, and O, as for example 1,2-methylenedioxybenzene.

The term “carboxyl,” as used herein, refers to a radical or substituent of the formula —C(═O)OH.

The term “carbonyl”, whether used alone or with other terms, such as “alkoxycarbonyl”, denotes —C(═O)—.

The term “alkylcarbonyl,” as used herein, refers to a radical or substituent of the formula —C(═O)-alkyl, and includes, for example, methylcarbonyl, ethylcarbonyl, propylcarbonyl, butylcarbonyl, and pentylcarbonyl.

The term “alkoxycarbonyl,” as used herein, refers to a radical or substituent —C(═O)—O-alkyl, and includes, for example, methoxycarbonyl, ethoxycarbonyl, propoxycarbonyl, butoxycarbonyl, and pentoxycarbonyl.

The terms “halogen” or “halo” which are used interchangeably herein, refer to a radical or substituent selected from the group consisting of chloro, bromo, iodo, and fluoro.

The term “polyhaloalkyl,” as used herein, refers to an alkyl radical or substituent having one or more halogen substituents and includes perhaloalkyl groups. Examples include trifluoromethyl, trifluoroethyl, and chlorodifluoromethyl.

The term “tautomeric form” as used herein refers two or more isomeric structures formed by migration of a hydrogen atom.

The term “amino” as used herein refers to an —NH₂ group.

The term “living host” as used herein refers to an organism that is living and capable of being infected with a virus, such as the hepatitis C virus; for example, a mammal, which includes a human.

As used herein, “hepatitis C virus” or “HCV” refers to any representative of a diverse group of related viruses classified within the hepacivirus genus of the Flaviviridae family.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

The term “NS4B” refers to a portion of the HCV genome located in the central portion of the viral genome that specifies the region encoding a protein, termed the “NS4B protein”, or “NS4B polypeptide”, or combinations of these terms which are used interchangeably herein. NS4B in its natural state, functions as a signal transducer which inhibits interferon stimulated gene expression, activates NF-κB pathways and enhances cellular proliferation. Thus, in accordance with the present invention it has been discovered that NS4B signaling modulates a variety of cellular processes including, without limitation, apoptosis, immune regulation and maintenance of the infected state in infected cells. The nucleic acid region encoding the NS4B protein may also be referred to as the “NS4B gene”. Thus, the term “NS4B” may refer to either a nucleic acid encoding the NS4B polypeptide, to an NS4B gene or to an NS4B polypeptide, or to any portions thereof, depending on the context in which the term is used. NS4B may further refer to natural allelic variants, mutants and derivatives of either NS4B nucleic acid sequences or NS4B polypeptides. The NS4B nucleic acid, NS4B gene or NS4B protein referred to may be either functional or non-functional. As set forth herein NS4B is a signal transducing molecule which functions to increase viral persistence. Certain compounds of the invention may be effective to inhibit NS4B action which is distinct from its signal transducing activity. Such activity includes without limitation the ability of NS4B to function as an anchor securing the replication complex to cellular membranes. The activity of such compounds can be determined by assessing viral replication in the presence and absence of the compound followed by determining the binding affinity of said compound for NS4B.

“Apoptosis” refers to a type of cell death that is thought to be under direct genetic control. During apoptosis, cells lose their cell junctions and microvilli. The cytoplasm condenses and nuclear chromatin marginates into a number of discrete masses. While the nucleus fragments, the cytoplasm contracts and mitochondria and ribosomes become densely compacted. After dilation of the endoplasmic reticulum and its fusion with the plasma membrane, the cell breaks up into several membrane bound vesicles, also known as apoptotic bodies, which are usually phagocytosed by adjacent cells. A compound having NS4B signal transducing inhibitory activity should be effective to induce NS4B expressing cells (which exhibit reduced apoptosis) to undergo programmed cell death. The efficacy of such compounds can be assessed by measuring alterations in apoptosis levels in the presence and absence of the compound. Optionally the binding affinity or binding constant of the compound for NS4B can be determined.

The present invention also includes active portions, fragments, derivatives and functional or non-functional mimetics of HCV NS4B polypeptides or proteins. An “active portion” of HCV NS4B polypeptide means a peptide that is less than the full length HCV NS4B polypeptide, but which retains measurable biological activity.

A “fragment” or “portion” of the HCV NS4B polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids.

A “derivative” of the HCV NS4B polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, and may or may not alter the essential activity of original the HCV NS4B polypeptide.

The HCV NS4B polypeptide or protein described herein also includes any variant which is derived from a HCV NS4B polypeptide and which retains at least one property or other characteristic of the HCV NS4B polypeptide. Different “variants” of the HCV NS4B polypeptide exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to the HCV NS4B polypeptide, (c) variants in which one or more amino acids include a substituent group, and (d) variants in which the HCV NS4B polypeptide is fused with another peptide or polypeptide such as a fusion partner, a protein tag or other chemical moiety, that may confer useful properties to the HCV NS4B polypeptide, such as, for example, an epitope for an antibody, a polyhistidine sequence, a biotin moiety and the like. Other HCV NS4B polypeptides of the invention include variants in which amino acid residues from one species are substituted for the corresponding residue in another species, either at the conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to the person having ordinary skill in the art. To the extent such allelic variations, analogues, fragments, derivatives, mutants, and modifications, including alternative nucleic acid processing forms and alternative post-translational modification forms result in derivatives of the HCV NS4B polypeptide that retain any of the biological properties of the HCV NS4B polypeptide, they are included within the scope of this invention.

The term “functional” as used herein implies that the nucleic or amino acid sequence is functional for the recited assay or purpose.

The phrase “consisting essentially of when referring to a particular nucleotide or amino acid means a sequence having the properties of a given SEQ ID No:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded. A sub-genomic replicon as used herein may refer to a nucleic acid construct which expresses the non-structural proteins of HCV and is expressed in Huh-7 cells. HCV replicons can be obtained from APATH, LLC (St. Louis, Mo.).

An “HCV replicon active” is a compound that inhibits replication of the HCV mini-genome of the HCV replicon system.

The replicon activity may be detected in a HCV replicon assay by measuring any number of signals related to HCV replication, for example by protein expression or by measuring RNA levels. Preferred methods of measuring viral protein expression include ELISA and Western Blot. A preferred method of measuring viral RNA levels is RT-PCR and a further preferred method of measuring RNA levels is through quantitative RT-PCR, for example by TaqMan. A representative example of a replicon assay is described in Example 1, below.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

All amino-acid residue sequences represented herein conform to the conventional left-to-right amino-terminus to carboxy-terminus orientation.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

“Mature protein” or “mature polypeptide” shall mean a polypeptide possessing the sequence of the polypeptide after any processing events that normally occur to the polypeptide during the course of its genesis, such as proteolytic processing from a polyprotein precursor. In designating the sequence or boundaries of a mature protein, the first amino of the mature protein sequence is designated as amino acid residue 1. In the case of the mature NS4B protein, its normal biogenesis entails its proteolytic cleavage from a precursor polyprotein.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, to that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radioimmunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other manners, the introduced nucleic acid may exist in the recipient cell or host organism only transiently.

A “clone” or “clonal cell population” is a population of cells derived from a single cell or common ancestor by mitosis.

A “cell line” is a clone of a primary cell or cell population that is capable of stable growth in vitro for many generations.

A “viral antigen” shall be any peptide, polypeptide or protein sequence, segment or epitope that is derived from a virus that has the potential to cause a functioning immune system of a host to react to said viral antigen.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunloglobulin molecule such as those portions known in the art as Fab, Fab′, F(ab′)2 and F(v). Compounds described herein are also useful in preventing or resolving viral infections in cell, tissue or organ cultures and other in vitro applications. For example, inclusion of compounds of the invention as a supplement in cell or tissue culture growth media and cell or tissue culture components will prevent viral infections or contaminations of cultures not previously infected with viruses. Compounds described above may also be used to eliminate and/or attenuate viruses from/in cultures or other biological materials infected or contaminated with viruses (for example, blood), after a suitable treatment period, under any number of treatment conditions as determined by the skilled artisan.

Compounds of the invention can form useful salts with inorganic and organic acids such as hydrochloric, sulfuric, acetic, lactic, or the like and with inorganic or organic bases such as sodium or potassium hydroxide, piperidine, ammonium hydroxide, or the like. The pharmaceutically acceptable salts of the compounds of Formula I are prepared following procedures that are familiar to those skilled in the art.

The isomeric forms of the compounds of the invention include, without limitation, the various isomers of the heterocyclic substituents that may be present therein. The chemical structures depicted herein and therefore the compounds of the invention also encompass all of the corresponding possible tautomeric forms. Such tautomers may, in certain instances, be resolved into individual compounds by methods known to those of skill in the art.

When used for treatment or prophylaxis of infection, the compounds described herein may be administered as such, or in the form of a pharmaceutical composition comprising one or more compounds of Formulas I-VIII above, as the active agent, and optionally at least one supplemental active ingredient, in combination with a pharmaceutically acceptable carrier medium or auxiliary agent.

A composition comprising a compound of the invention may be prepared in various forms for administration, including tablets, caplets, pills or dragees, or can be filled in suitable containers, such as capsules, or, in the case of suspensions, filled into bottles. As used herein, “pharmaceutically acceptable carrier medium” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Twentieth Edition, A. R. Gennaro (William and Wilkins, Baltimore, Md., 2000) discloses various carriers used in formulating pharmaceutical compositions and known techniques for the preparation thereof. Except insofar as any conventional carrier medium or auxiliary agent is incompatible with the antiviral compounds of the invention, such as by producing any undesirable biological effect or otherwise interacting in a deleterious manner with any other component(s) of the pharmaceutical composition, its use is contemplated to be within the scope of this invention.

In the pharmaceutical compositions of the invention, the active agent may be present in an amount of at least 0.5% and generally not more than 90% by weight, based on the total weight of the composition, including carrier medium and/or auxiliary agent(s), if any. Preferably, the proportion of active agent varies between 5 to 50% by weight of the composition.

Pharmaceutical organic or inorganic solid or liquid carrier media suitable for enteral or parenteral administration can be used to make up the composition. Gelatine, lactose, starch, magnesium stearate, talc, vegetable and animal fats and oils, gum, polyalkylene glycol, or other known medicament components may all be suitable as carrier media or excipients.

The compounds of the invention may be administered using any amount and any route of administration effective for attenuating infectivity of the virus. Thus, the expression “therapeutically effective amount,” as used herein, refers to a nontoxic but sufficient amount of the antiviral agent to provide the desired prophylaxis and/or treatment of viral infection. The exact amount required will vary from subject to subject, depending on the species, age, and general condition of the subject, the severity of the infection, the particular antiviral agent, its mode of administration, and the like.

The antiviral compounds are preferably formulated in dosage unit form for ease of administration and uniformity of dosage. “Dosage unit form” as used herein refers to a physically discrete unit of antiviral agent appropriate for the patient to be treated. Each dosage should contain the quantity of active material calculated to produce the desired therapeutic effect either as such, or in association with the selected pharmaceutical carrier medium and/or the supplemental active agent(s), if any. Typically, the antiviral compounds of the invention will be administered in dosage units containing from about 2 mg to about 7000 mg of the antiviral agent by weight of the composition, with a range of about 10 mg to about 2000 mg being preferred.

The compounds may be administered orally, rectally, parenterally, such as by intramuscular injection, subcutaneous injection, intravenous infusion or the like, intracisternally, intravaginally, intraperitoneally, locally, such as by powders, ointments, or drops, or the like, or by inhalation, such as by aerosol or the like, taking into account the nature and severity of the infection being treated. Depending on the route of administration, the compounds of the invention may be administered at dosage levels of about 0.05 to about 100 mg/kg of subject body weight per day, one or more times a day, to obtain the desired therapeutic effect.

The compounds of the invention will typically be administered from 1 to 4 times a day so as to deliver the above-mentioned daily dosage. However, the exact regimen for administration of the compounds and compositions described herein will necessarily be dependent on the needs of the individual host or patient being treated, the type of treatment administered and the judgment of the attending medical specialist.

In view of the inhibitory effect on viral RNA replication produced by the compounds exemplified below, it is anticipated that these compounds will be useful not only for therapeutic treatment of virus infection, but for virus infection prophylaxis, as well. The dosages may be essentially the same, whether for treatment or prophylaxis of virus infection.

As previously mentioned, the compounds of the present invention, their isomeric forms and pharmaceutically acceptable salts thereof are useful, per se, in treating and preventing viral infections, in particular hepatitis C infection, and diseases in living hosts, or in combination with each other, or with supplemental biologically active agents, including but not limited to the group consisting of interferon, a pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds. Such combination therapy may also comprise providing a compound of the invention either concurrently or sequentially with other medicinal agents or potentiators, such as acyclovir, famicyclovir, valgancyclovir and related compounds, ribavirin and related compounds, amantadine and related compounds, various interferons such as, for example, interferon-alpha, interferon-beta, interferon-gamma and the like, as well as alternative forms of interferons such as pegylated interferons. Additionally, combinations of, for example ribavirin and interferon, may be administered as an additional combination for a multiple combination therapy with at least one of the compounds of the present invention.

The combination therapy can be sequential, that is the treatment with one agent first and then the second agent (for example, where each treatment comprises a different compound of the invention or where one treatment comprises a compound of the invention and the other comprises one or more biologically active agent), or it can be treatment with both agents at the same time (concurrently). The sequential therapy can be within a reasonable time after the completion of the first therapy before beginning the second therapy. The treatment with both agents at the same time can be in the same daily dose or in separate doses. The dosages for both concurrent and sequential combination therapy will depend on absorption, distribution, metabolism, and excretion rates of the components of the combination therapy as well as other factors known to one of skill in the art. Dosage values will also vary with the severity of the condition to be alleviated. It is to be further understood that for any particular subject, specific dosage regimens and schedules may be adjusted over time according to the individual's need and the professional judgment of the person administering or supervising the administration of the combination therapy.

In a further embodiment, the compounds of the present invention may be used for the treatment of HCV in humans in combination therapy mode with other inhibitors of the HCV life cycle such as, for example, inhibitors of HCV cell attachment or virus entry, HCV translation, HCV RNA transcription or replication, HCV maturation, assembly or virus release, or inhibitors of HCV enzyme activities such as the HCV nucleotidyl transferase, helicase, protease or polymerase.

It is intended that combination therapies of the present invention include any chemically compatible combination of a compound of this inventive group with other compounds of the inventive group or other compounds outside of the inventive group, as long as the combination does not eliminate the anti-viral activity of the compound of this inventive group or the anti-viral activity of the pharmaceutical composition itself.

The term “interferon-alpha” as used herein means the family of highly homologous species-specific proteins that inhibit viral replication and cellular proliferation and modulate immune response. Typical suitable interferon-alphas include, but are not limited to, recombinant interferon alpha-2b such as INTRON-A INTERFERON available from Schering Corporation, Kenilworth, N.J., recombinant interferon alpha-2a such as Roferon interferon available from Hoffmann-La Roche, Nutley, N.J., a recombinant interferon alpha-2C, such as BEROFOR ALPHA 2 INTERFERON available from Boehringer Ingelheim Pharmaceutical, Inc., Ridgefield, Conn., interferon alpha-n1, a purified blend of natural alpha interferons such as SUMIFERON available from Sumitomo, Japan or as Wellferon interferon alpha-n1 (INS) available from Glaxo-Wellcome Ltd., London, Great Britain, or a consensus alpha interferon such as those described in U.S. Pat. Nos. 4,897,471 and 4,695,623 (the contents of which are hereby incorporated by reference in their entireties, specifically examples 7, 8 or 9 thereof) and the specific product available from Amgen, Inc., Newbury Park, Calif., or interferon alpha-n3 a mixture of natural interferons made by Interferon Sciences and available from the Purdue Frederick Co., Norwalk, Conn., under the ALFERON trademark. The use of interferon alpha-2a or alpha 2b is preferred. Since interferon alpha 2b, among all interferons, has the broadest approval throughout the world for treating chronic hepatitis C infection, it is most preferred. The manufacture of interferon alpha 2b is described in U.S. Pat. No. 4,503,901.

The term “pegylated interferon” as used herein means polyethylene glycol modified conjugates of interferon, preferably interferon alpha-2a and alpha-2b. The preferred polyethylene-glycol-interferon alpha-2b conjugate is PEG.sub.12000-interferon alpha 2b. The phrase “PEG.sub.12000-IFN alpha” as used herein means conjugates such as are prepared according to the methods of International Application No. WO 95/13090 and containing urethane linkages between the interferon alpha-2a or alpha-2b amino groups and polyethylene glycol having an average molecular weight of 12000.

The compounds of this invention may be prepared in general by methods known to those skilled in the art.

Representative examples of compounds of the invention are set forth in Tables 1-8 below, which list a series of compounds of Formulas I-VIII and provide data regarding their ability to inhibit HCV replication as assessed by NS5A proteins levels. Any of the nonstructural proteins are suitable for use in this assay. NS5A was chosen because it has the shortest half life of the NS proteins of HCV.

TABLE 1
Phenyl Benzamide Compounds:

| | | | ELISA | CV |
|---|---|---|---|---|
| Example | | Kd | EC50 | CC₇₅ |
| Number | Compound | (μM) | (μM) | (μM) |
| 1 | 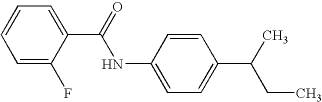 | 2.531 | 2.7 | 12 |
| 2 | 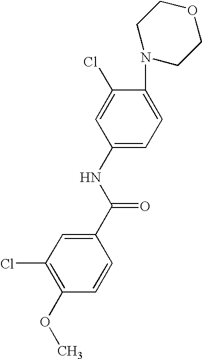 | 2.324 | 2.5 | 12 |
| 3 | 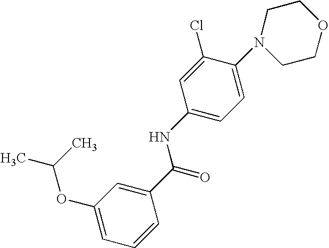 | 4.434 | 3.9 | 15 |
| 4 | 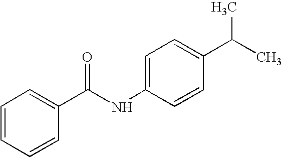 | 6.902 | 2.8 | 100 |

TABLE 2
Pyrazolopyrimidine Compounds:

| | | | ELISA | |
|---|---|---|---|---|
| Example | | Kd | EC50 | |
| Number | Compound | (μM) | (μM) | CV CC₇₅ |
| 1 | 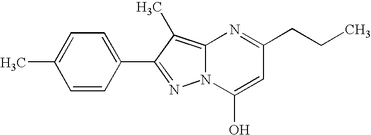 | 5.955 | 2.2 | 100 |
| 2 | 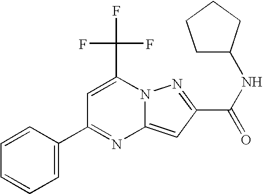 | 4.188 | 1.7 | >100 |
| 3 | 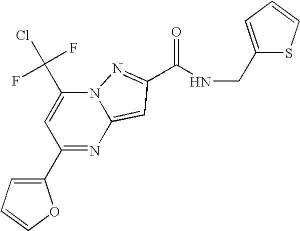 | 0.662 | 0.63 | >100 |

TABLE 3
Trifluoromethylpyrimidine Compound:

| | | | ELISA | |
|---|---|---|---|---|
| Example | | Kd | EC50 | CV |
| Number | Compound | (μM) | (μM) | CC₇₅ |
| 1 | 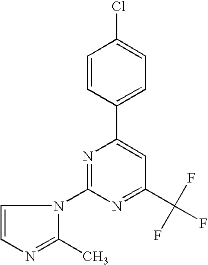 | 2.057 | 2.2 | >100 |

TABLE 4
Thienopyrazole Compound:

| | | | ELISA | |
|---|---|---|---|---|
| Example | | Kd | EC50 | |
| Number | Compound | (μM) | (μM) | CV CC₇₅ |
| 1 | 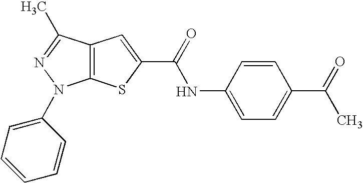 | 1 | 3.15 | 50 |

TABLE 5
Aminothiophene Compounds:

| | | | ELISA | |
|---|---|---|---|---|
| Example | | Kd | EC50 | CV |
| Number | Compound | (μM) | (μM) | CC₇₅ |
| 1 | 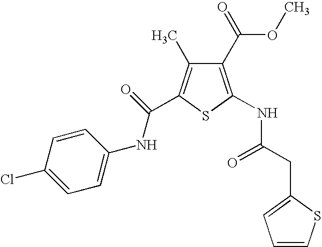 | 0.304 | 0.7 | 75 |
| 2 | 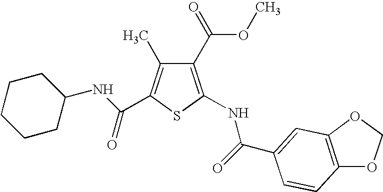 | 4 | 7.6 | 75 |
| 3 | 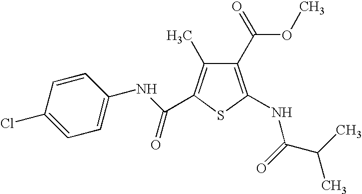 | 0.842 | 1.15 | 65 |
| 4 | 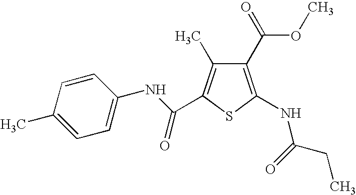 | 1.673 | 3.2 | >100 |
| 5 | 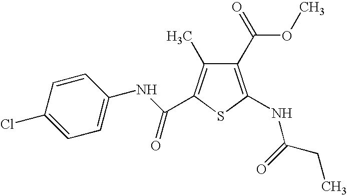 | 2.52 | 4.55 | >100 |

TABLE 6
Phenylthiazolylamine Compounds:

| | | | ELISA | |
|---|---|---|---|---|
| Example | | Kd | EC50 | CV |
| Number | Compound | (μM) | (μM) | CC₇₅ |
| 1 | 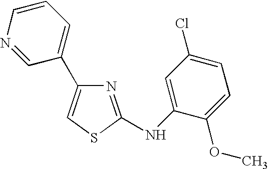 | 1.7 | 1.5 | 50 |
| 2 | 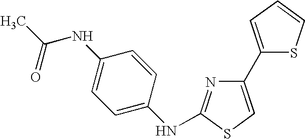 | 6.2 | 11.2 | 13 |
| 3 | 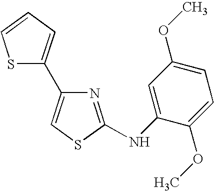 | 0.57 | 2.5 | 9 |

TABLE 7
Triazinoindole Compounds:

| | | | ELISA |
|---|---|---|---|
| Example | | Kd | EC50 |
| Number | Compound | (μM) | (μM) |
| 1 | 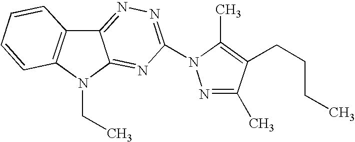 | 1.2 | 1.13 |
| 2 | 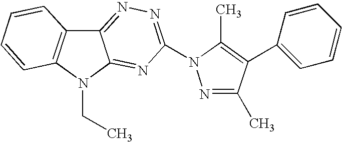 | 2.2 | 1.0 |
| 3 | 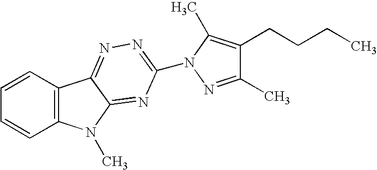 | 4.3 | 5.8 |
| 4 | 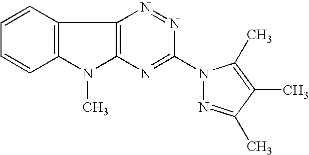 | 14 | >25 |
| 5 | 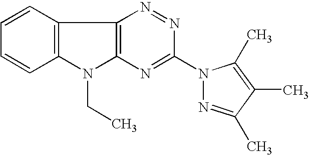 | 15.6 | >25 |
| 6 | 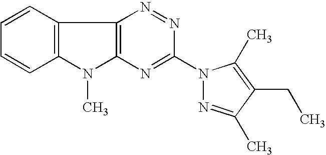 | 7.5 | >25 |
| 7 | 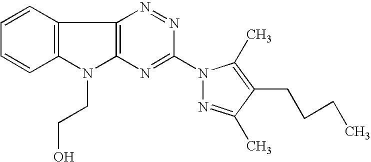 | 2 | >25 |
| 8 | 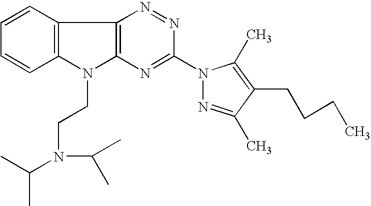 | 10.3 | 6.55 |
| 9 | 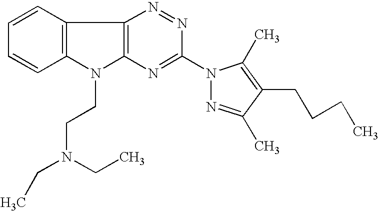 | 1.8 | 5.2 |
| 10 | 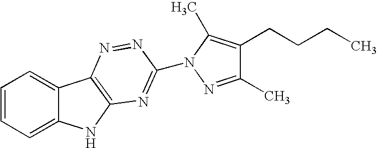 | 3.6 | >25 |
| 11 | 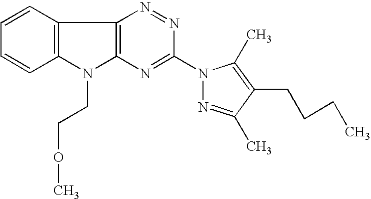 | 1 | 7 |
| 12 | 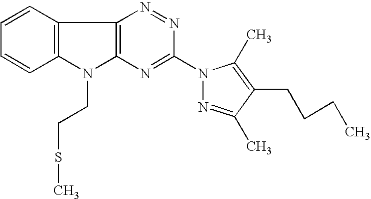 | 1.8 | 10 |

TABLE 8
Tetrahydrobenzothiophene Compound

| | | | ELISA |
|---|---|---|---|
| Example | | Kd | EC50 |
| Number | Compound | (μM) | (μM) |
| 1 | 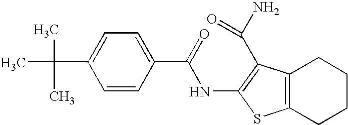 | 0.311 | 0.87 |

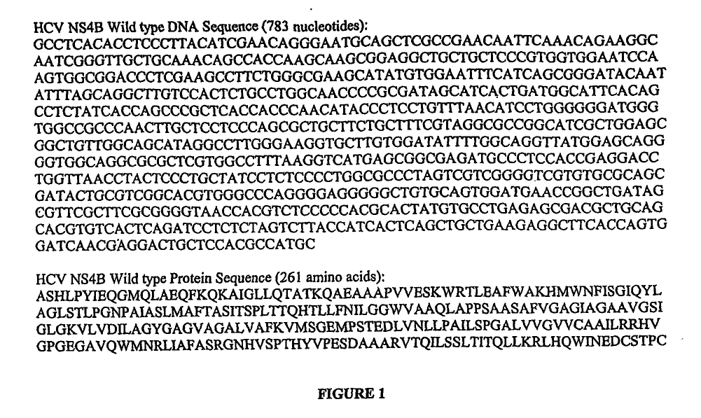

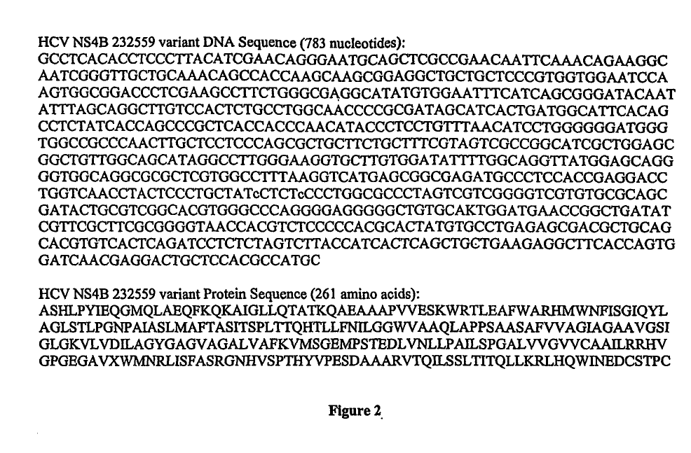

Methods for assaying the antiviral activity of candidate compounds frequently entail recombinant expression of functional viral proteins. FIG. 1 provides nucleic acid and amino acid sequences for this purpose. As mentioned previously, variants of the HCV genome are prevalent in nature. FIG. 2 provides functional variants of NS4B encoding nucleic acids and the corresponding variant amino acid sequence. Expression of recombinant HCV NS4B gene sequences may be carried out in a variety of systems including but not limited to bacterial, yeast, mammalian, insect and plant cell systems, as well as in organisms such as infected, transfected, transduced or transgenic insects, animals or plants. In one embodiment of the invention, HCV replicons were obtained which express HCV NS4B gene sequences in Huh-7 cells following transfection in culture.

The availability of nucleic acids molecules encoding HCV NS4B protein enables production of the protein using in vitro expression methods known in the art. For example, a cDNA or gene may be cloned into an appropriate in vitro transcription vector, such as pSP64 or pSP65 for in vitro RNA synthesis, followed by cell-free translation of the RNA in a suitable cell-free translation system, such as extracts of wheat germ, rabbit reticulocytes or HeLa cells. In vitro transcription and translation systems are commercially available (e.g., Promega Biotech, Madison, Wis.; Gibco-BRL, Gaithersburg, Md.).

Alternatively, according to a preferred embodiment of the invention, larger quantities of HCV NS4B protein may be produced by expression in suitable prokaryotic or eukaryotic systems such as bacterial, fungal, mammalian or plant systems. For example, part or all of a DNA molecule, such as a cDNA may be inserted into a plasmid vector adapted for expression in a bacterial cell, such as E. coli, or into a baculovirus vector for expression in an insect cell. Such vectors comprise the regulatory elements necessary for expression of the DNA in the host cell (e.g., E. coli or insect cell), positioned in such a manner as to permit expression of the DNA in the host cell. Such regulatory elements required for expression may include promoter sequences, transcriptional initiation and termination sequences, enhancer sequences, translational control sequences and the like.

The HCV NS4B proteins or derivatives thereof produced by gene expression in a recombinant prokaryotic or eukaryotic system may be purified according to methods known in the art In one embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein from extracts of expressing cells, tissues or organs by standard protein purification techniques or by affinity separation techniques, such as by immunological interaction with antibodies that bind specifically to the recombinant protein or by nickel columns for isolation of recombinant proteins tagged with 5-8 histidine residues at their N-terminus or C-terminus. Such methods are commonly used by skilled practitioners.

The HCV NS4B proteins of the invention, prepared by the aforementioned methods, may be analyzed according to standard procedures. For example, such proteins may be subjected to electrophoretic analyses and to amino acid sequence analyses, as well as to crystallographic analyses for structure determination according to known methods. Such analyses provide useful information regarding the functionality of the NS4B protein and on means to affect that functionality, such as in the design of molecules that may inhibit the function of the NS4B protein.

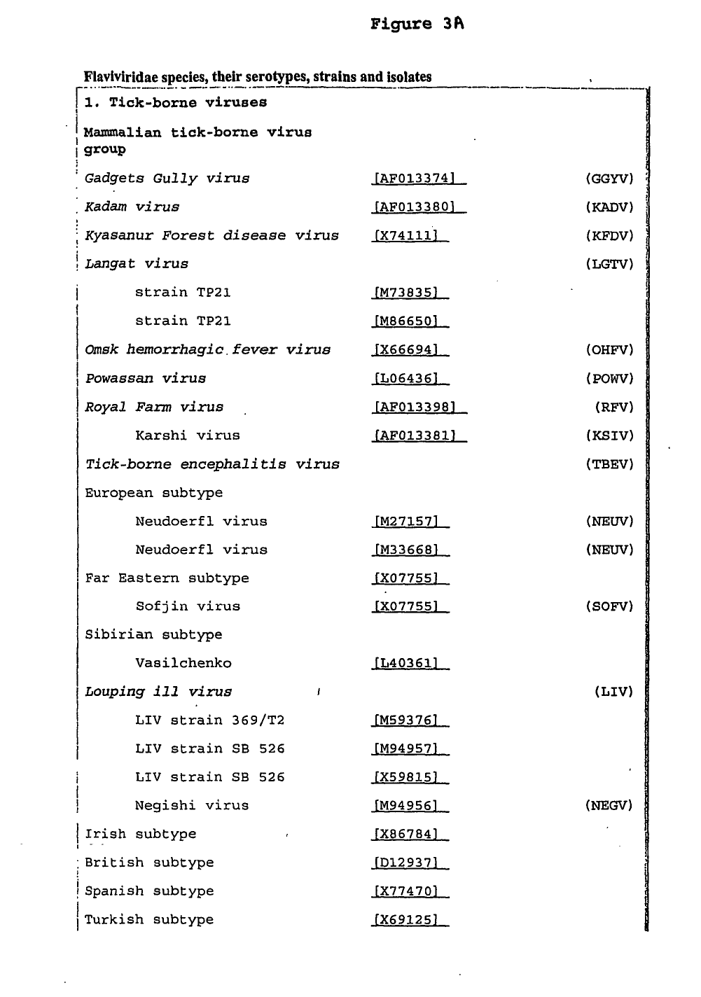

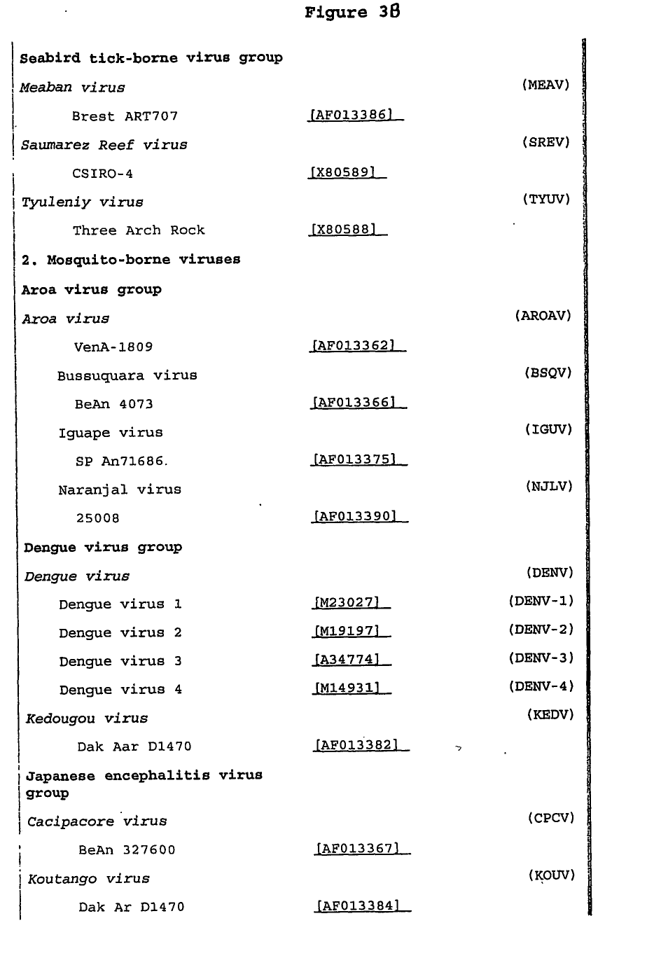

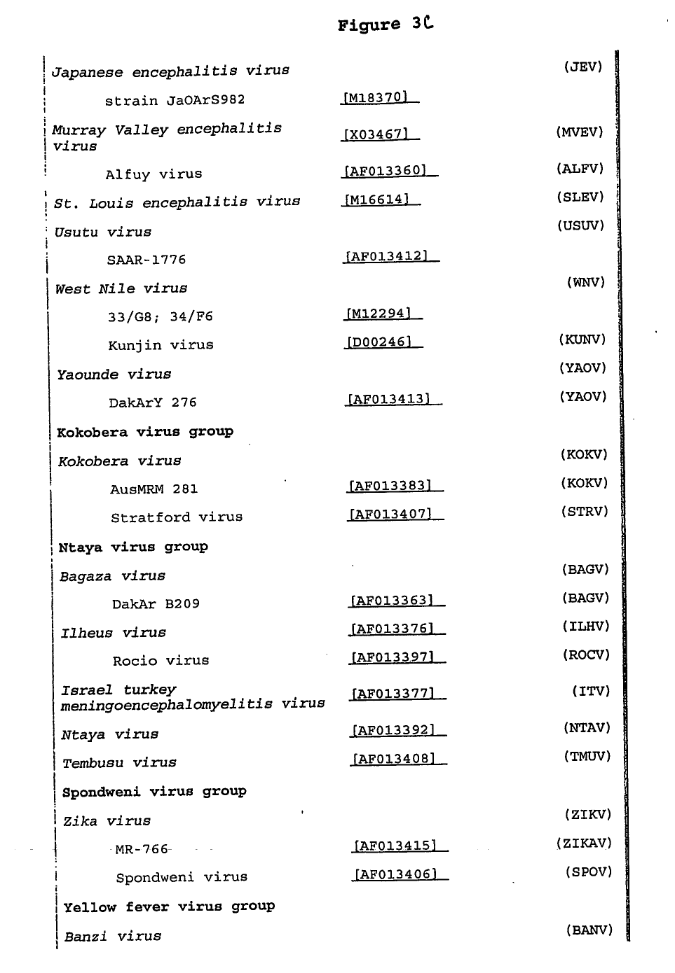

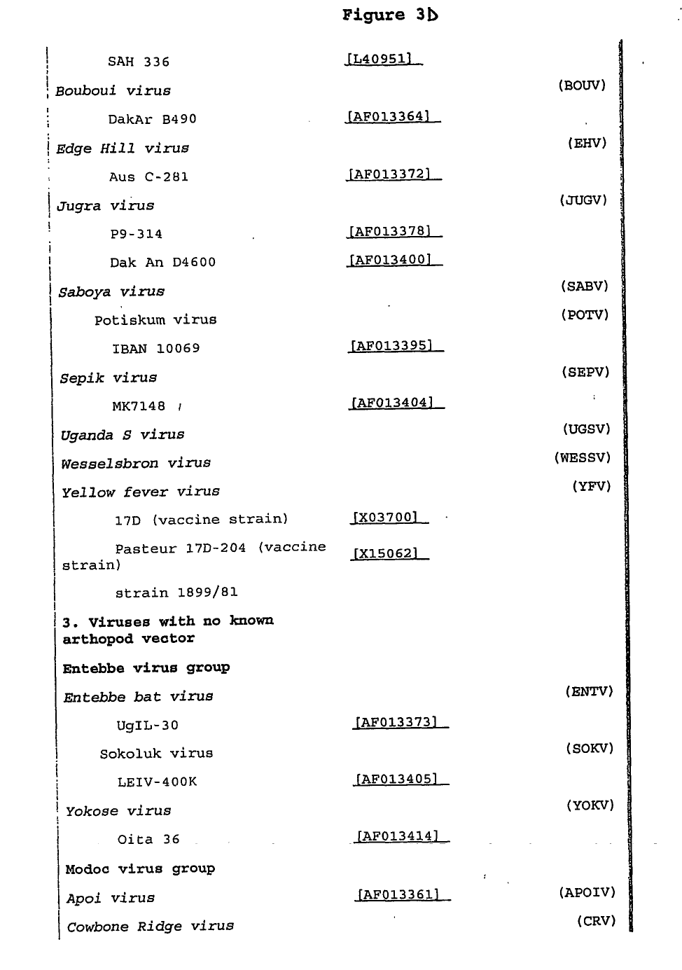

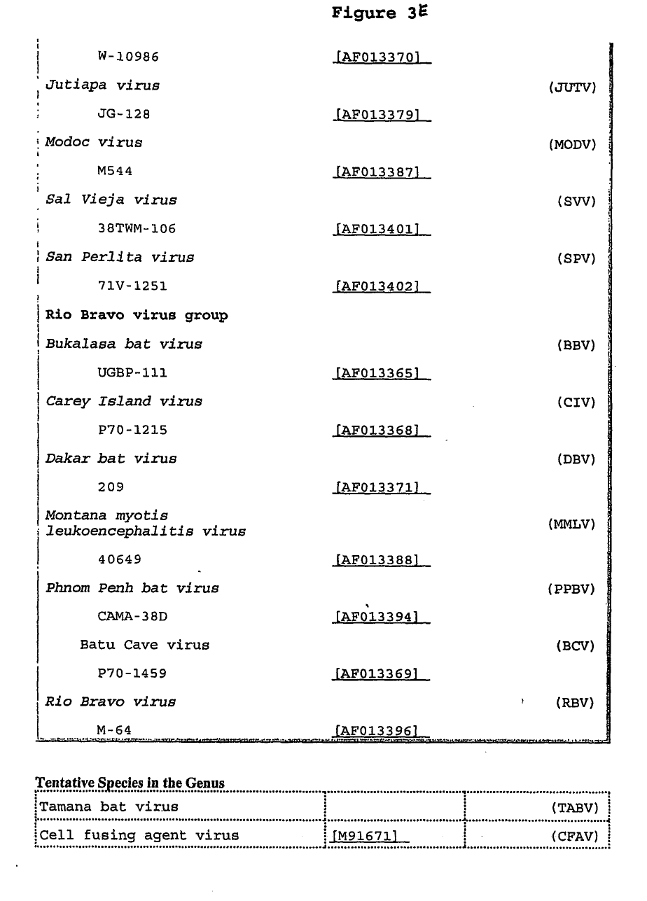

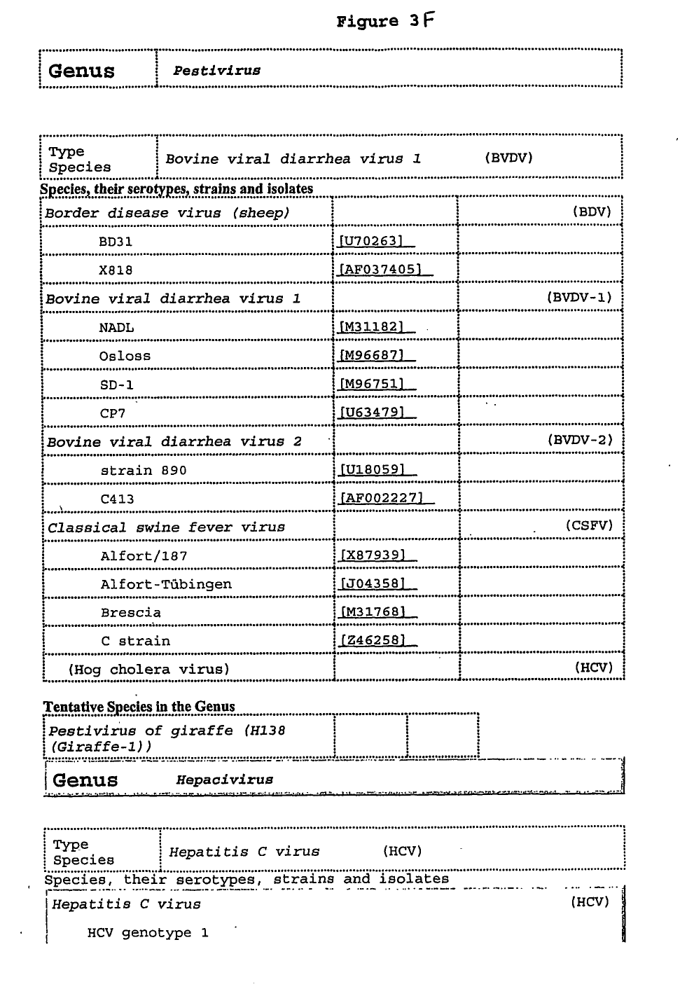

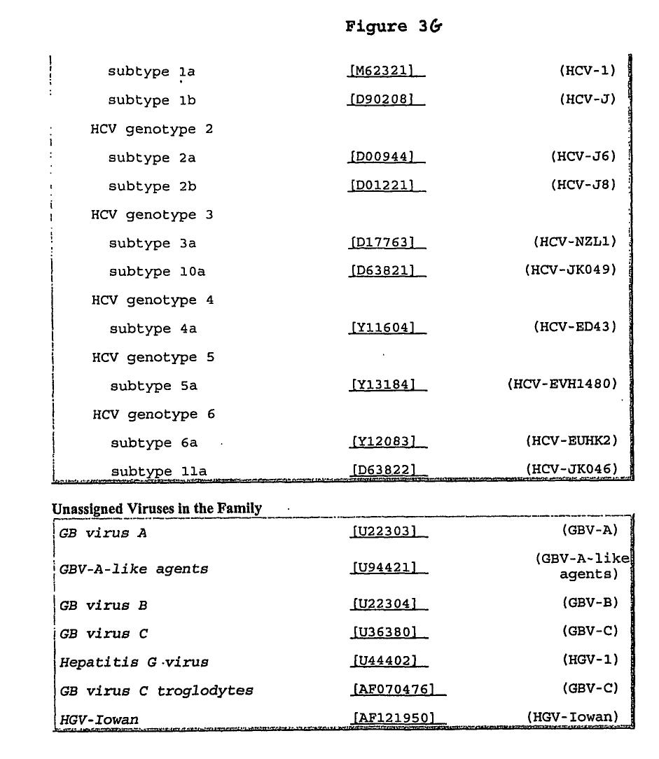

Again, while NS4B protein from HCV is exemplified herein, other viruses possess NS4B like proteins. FIG. 3 provides a list of these viruses and the GenBank accession numbers which provide the sequence information therefore. The skilled artisan can readily isolate “homologs” of HCV NS4B from these viral sequences and assay the instantly claimed compounds for efficacy as antiviral agents as disclosed herein.

The assay methods of the invention can be designed such that the aforementioned protein sequences are provided and then contacted with agents or materials suspected of interacting with such sequences and the effect of such agents on HCV NS4B activity is measured. The affect of such agents on the HCV NS4B activity may be measured in any number of ways. For example, NS4B associated apoptosis that is directly or indirectly dependent on the HCV NS4B activity may be quantified in the presence and absence of a test compound. Methods for analyzing apoptosis are well known to the skilled person. Agents identified in such interaction assays would have potential diagnostic utility involving modulation of NS4B associated apoptosis. Such agents would also have potential utility in applications involving the prevention or treatment of HCV disease in an affected living host, including humans, and for the inhibition or enhancement of HCV replication or propagation in living hosts and in in vitro systems such as cell, tissue and organ cultures.

Candidate therapeutic agents exposed to NS4B protein may be assessed for their ability to specifically affect apoptosis modulating activity. Such active NS4B may be provided in an extract or lysate of a cell in which the polypeptide was produced, in an in vitro cell-free expression system or in an enriched or purified form.

There are numerous means by which the apoptosis modulating activity of the HCV NS4B protein provided in an extract, cell-free system or enriched form may be assessed, and these are well known in the art. Kits for detection of apoptosis are available from Sigma and include cytochrome c oxidase assay kits (CTOX-OX1), apoptosis PCR Bax/Bcl2 multiplex primer sets(APO-PCR), terminal transferase from calf thymus (T4427) and Triosalen (used as a probe for nucleic acid structure and function, T6137). Promega provides the TUNEL assay.

Assays involving the nucleic acid and polypeptide compositions of the invention may be formatted in any number of configurations. Particularly useful for evaluating large numbers of agents and materials are high throughput screening formats. Traditionally such assays were typically formatted in 96 well plates. However, 384, 864 and 1536 well plates may be used in such high throughput assay systems. These systems are often automated using robotics technologies to allow manipulation and processing of large numbers of samples.

The agents or materials that may be evaluated in the various assay methods of the invention for potential antagonistic or agonistic affects include but are not limited to small molecules, such as those of Formula I and Formula II, polymers, peptides, polypeptides, proteins, immunoglobulins or fragments thereof, oligonucleotides, antisense molecules, peptide-nucleic acid conjugates, ribozymes, polynucleotides and the like. The potential utility of agents or materials identified using the compositions and assay methods of the invention will be broad and will include uses for the detection and isolation of HCV nucleic acids and polypeptides, for the detection or diagnosis of HCV, for the prevention and treatment of HCV disease in an affected living host, including humans, and for the inhibition or enhancement of HCV replication or propagation in living hosts and in in vitro systems such as cell, tissue and organ cultures, as well as for other uses that may be envisioned once the nature of the agent is clear.

The following examples are provided to describe the invention in further detail. These examples, which set forth the preferred mode presently contemplated for carrying out the invention, are intended to illustrate and not to limit the invention.

### EXAMPLE 1

### Inhibition of Viral RNA Replication

Antiviral activity of representative compounds of the invention was evaluated in a human liver-derived cell line (Huh-7-Clone A) containing the HCV replicon (BB7 sequence) (See Lohmann et al. Science. 1999, 285:110-3; Blight K J et al., Science. 2000, 290:1972-4; Pietschmann, T. et al., J. Virol. 2001, 73:1252-1264; and Lohmann, V. et al., J. Virol. 2001, 75:1437-1449). The HCV replicon is a subgenomic viral RNA that expresses the HCV proteins required for its own replication. These proteins include non-structural proteins NS3, NS4A, NS4B, NS5A and NS5B. The replicon also contains a foreign gene encoding a drug-selectable marker (neomycin phosphotransferase) to allow for G418 (neomycin) selection of cells that contain the replicon.

An ELISA (enzyme-linked immunosorbant assay) was used to determine the effect of compounds within the scope of the invention on the amount of HCV NS5A protein produced after a 72-hour incubation of the replicon-containing cells in the presence of varying concentrations of compound. Huh7-Clone A cells were seeded in 96-well plates at a subconfluent density (9000 cells/well) in medium containing 2% FBS and incubated for 4 hours to allow attachment to occur. HCV-086 (solubilized with 100% dimethylsulfoxide [DMSO]) was added to wells using an 8-point, 3-fold serial dilution series, with a final DMSO concentration of 1% in a total volume of 200 μL. Plates were incubated for 72 hours at 37° C. and 5% CO₂. Under these conditions, the cells are approximately 25% confluent at the time of seeding and 80-90% confluent on day 3. COSTAR® 96-well cell culture plates were used but other known cell culture plates may be used. After incubation, media is removed from wells and the cells are fixed to the assay plate using 0.05% glutaraldehyde (Fisher # 02957-4). The glutaraldehyde is then washed off using phosphate-buffered saline (PBS) following a 1 hour incubation and cells are blocked for non-specific antibody binding using for example SUPERBLOCK® reagent (blocking buffer) in PBS. The blocking agent is rinsed from the cells with PBS after 1 hour at 37° C., and HCV NS5A monoclonal antibody (Virostat # 1873) is added to each well containing compound. Primary antibody is incubated for 1 hour at 37° C. and rinsed 3 times with PBS containing 0.02% TWEEN-20™ before addition of Horseradish Peroxidase (HRP) conjugated secondary antibody. HRP is incubated for 1 hour at 37° C. and rinsed several times, first with PBS/TWEEN-20™ followed by PBS alone. To quantify peroxidase activity, 3,3′,5,5′-tetramethylbenzidine (TMB) substrate is added to the plate and after 30 minutes, the plates are read in an ELISA plate reader at an OD of 650 nm.

Compound dose response was measured in an 8-point dose curve diluted serially to determine the inhibitory concentration at 50% (EC₅₀ value). Representative compounds of the invention showed a dose-dependent inhibition of intracellular NS5A levels. Ranges of 50% effective concentrations (EC₅₀s) for the representative compounds within the scope of this invention are listed in Tables 1-8. Preferred compounds have 50% effective concentrations at about 30 μM or less, more preferred compounds have 50% effective concentrations at about 5 μM or less, and most preferred compounds have 50% effective concentrations at about 0.5 μM or less.

### EXAMPLE 2

### NS4B Binding Assay Protocol

NS4B was cloned, expressed and purified to establish an assay, which takes advantage of the intrinsic fluorescent properties of proteins and identifies compounds, which inhibit by a NS4B specific mechanism. This protein is necessary for viral replication. It is thought to act as an anchor securing the replication complex to cellular membranes, where replication is known to occur. Several other functions necessary for viral replication have also ascribed to NS4B. In this assay, the quenching of the intrinsic protein fluorescence upon binding (λₑₓ=280 nm; λₑₘ=330 nm) to a small molecule was quantitated at different concentrations of the ligand, and the plot of fluorescence quenching as a function of ligand concentration provided the binding constant (K_{d}). The compounds described herein specifically quench NS4B protein fluorescence in a dose dependent manner. The K_{d} values of representative compounds of the invention are listed in Tables 1-8.

### EXAMPLE 3

### Method for Distinguishing Biological Activity From Cellular Cytotoxicity

The compounds of the invention which had been identified as having both replicon activity and which bind to NS4B were further evaluated to distinguish between observed anti-viral biological activity and chemical cytotoxicity according the following method.

Huh-7 cells (harboring no replicon) and wild type replicon cells were seeded onto a 96 well plate. All cells were subjected to increasing amounts of a representative compound of the invention, as described in the replicon ELISA protocol. In order to assess the cellular toxicity of a compound of Formula I in both Huh-7 cells (harboring no replicon) and wild type replicon, cells were stained with Crystal Violet (a nonspecific protein stain) after 72 hours. For these experiments the CC₇₅(CV) values for cells containing wild type replicons are listed in Tables 1-5. The CC₇₅ values for cells lacking replicon were generally >100.0 μM in parental Huh-7 cells for each compound. If a compound exhibits more cellular toxicity in cells comprising HCV replicons than in the parental Huh-7 cell line, then it is believed that the specific action of the compound when it binds to NS4B is to disrupt the anti-apoptotic effects exerted by NS4B. In this case the cells' natural defenses are strengthened and apoptosis, a natural defense to viral infection, occurs. The Huh-7 cell line is the parental cell line harboring no HCV subgenomic replicon. In this cell line, the toxicity is reduced due to the fact that there is no NS4B for the compound to bind. If the cellular toxicity in cells comprising HCV replicons and in the parental Huh-7 cell line (no replicon) are the same, then they are effective in inhibiting HCV replication rather than the modulation of apoptosis.

An additional specific-application of this method will now be described.

During drug screening efforts to identify compounds having binding affinity for HCV NS4B, the triazinoindole class of compounds of Formula VII was identified. When EC₅₀ was measured using the HCV subgenomic replicon system, a value of 1.2 μM was obtained for the compound shown as Example 1 in Table 7. However a cellular toxicity (CC₅₀) of 20 μM was also found. In order to determine the nature of this toxicity, Huh-7 cells (harboring no replicon) and wild type replicon cells were seeded onto a 96 well plate. All cells were subjected to increasing amounts of the triazinoindole compound, as described in the replicon ELISA protocol. Following a 72-hour period, cells were stained with Crysal Violet, a nonspecific protein stain, in order to assess the cellular toxicity of the test compound in the various cell lines. In these experiments, the CC₅₀ values in parental cells lacking replicon vs. cells containing wild type replicons were as follows: 20. 0 μM in cell containing wild type replicons and >100.0 μM in parental Huh-7 cells. Thus, the test compound exhibits more cellular toxicity in cells comprising HCV replicons than in the parental Huh-7 cell line. This phenomenon can be explained by the action of the compound on NS4B. In wild type cell lines, the test compound binds to NS4B, thereby disrupting the anti-apoptotic effects exerted by NS4B. In this case, the cells natural defenses are strengthened and apoptosis, a natural defense to viral infection, occurs. The Huh-7 cell line is the parental cell line harboring no HCV subgenomic replicon. In this cell line, the toxicity is reduced due to the fact that there is no NS4B for the triazinoindole compound to bind. Taken together these results indicate not only a NS4B specific mechanism of inhibition for triazinoindole molecules but also a regulatory function of HCV NS4B in the cellular apoptotic pathway.

To select for replicon variants, genotype 1b (BB7 isolate) replicon-containing cells were cultured and in the presence of 5 μM of the test compound for 7 passages. As a control, genotype 1b (BB7 isolate) replicon-containing cells were passaged in parallel, without the test compound. The cell line passaged in the presence of the test compound was found to have over 10-fold reduced susceptibility to the compound, while the control cell line had similar susceptibility. Total cellular RNA was extracted from both cell lines using Trizol Reagent (Invitrogen #15596-026) according to the manufacturer's protocol. NS5B cDNA was generated in a two-step RT-PCR reaction. PCR products were ligated into pPCR-Script Amp SK (+) (Stratagene #211188), transformed into E. coli, and sequenced.

The data show three changes in the NS4B region while no changes were observed in the control (cell line without the test compound). The changes are as follows: K52R, G120V and A210S. NS5B region was also sequenced and no amino acid changes were observed in either the cell line treated with the test compound or in the cell line without the compound. These results indicate that the compound specifically interacts with NS4B.

### EXAMPLE 4

### Analysis of Triazinoindole Cellular Toxicity Link to HCV Protein Expression in Huh7 Cells

2-(3,5-dimethyl-4-phenyl-pyrazol-1-yl)-9-methyl-9H-1,3,4,9-tetraaza-fluorene (compound A) and 2-(4-butyl-3,5-dimethyl-pyrazol-1-yl)-9-ethyl-9H-1,3,4,9-tetraaza-fluorene (compound B) are triazinoindole analogs of the invention, which demonstrated activity (as measured by Elisa and quantitative RT-PCR) against HCV-replicon using Clone-A cells. The activity of these compounds is believe to be mediated through binding to NS4B, as these compounds demonstrated binding activity towards purified HCV NS4B in a fluorescent binding assay. HCV NS4B is believed to play an anti-apoptotic role following HCV cellular infection. In order to determine if the cellular toxicity of the triazinoindole were mediated by HCV proteins such as NS4B, the following experiments were performed.

The cellular toxicity of Compound A at different concentrations was assessed on different cell lines following crystal violet staining (qualitative cell proliferation assay) after 5 day incubation. Huh7 cells are human hepatoma cells. A genotype 1b replicon-containing cell line derived from Huh7 cells (Clone-A) was obtained from Apath, LLC. Clone-A cells were selected under Compound B drug pressure and a resistant clone was selected (559^{R}) which contained mutations in NS4B gene. Compound A demonstrated more cellular toxicity on Clone-A cells (˜3 uM) compared to Huh-7 and 559^{R} cells (˜30 uM). This data suggest that Compound A toxicity is mediated by HCV proteins such as NS4B.

DNA fragmentation is a measure of apoptotic cell death. Huh7, Clone-A and 559^{R} were treated with Compound B and genomic DNA fragmentation determined by agarose gel electrophoresis. These data demonstrated that Compound B cause DNA fragmentation (apoptose) in Clone-A cells only suggesting that the cellular toxicity of Compound B is through apoptose in human cells expressing HCV proteins while though a different mechanism, and at higher Compound B concentration, in human hepatoma cells (Huh7) and in those expressing an HCV replicon resistant to Compound B (559^{R}). These data also suggest that Compound B toxicity in Clone-A cells is mediated by inhibition of NS4B functions.

Although certain preferred embodiments of the present invention have been described and/or exemplified above, various other embodiments will be apparent to those skilled in the art from the foregoing disclosure. The present invention is, therefore, not limited to the particular embodiments described and/or exemplified, but is capable of considerable modification without departure from the scope of the appended claims. For example, NS4B from HCV is exemplified herein; however, other related viral families possess NS4B proteins which are homologous and function in a manner comparable to HCV NS4B. Accordingly, the present invention encompasses methods for identification and use of agents which modulate NS4B function from such related viruses which include, but are not limited to, flaviviruses, pestiviruses and additional hepaciviruses.

What is claimed is:

1. A composition for prophylaxis or treatment of viral infections, said composition comprising a therapeutically effective amount of a compound, including pharmaceutically acceptable salts thereof, selected from the group of those having the formulas: 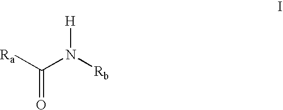
wherein:
Rₐ represents a radical selected from the group consisting of cycloalkyl, a heterocyclic radical, a substituted or unsubstituted aryl group, and a substituted or unsubstituted heteroaryl group;
R_{b} represents a radical selected from the group consisting of a substituted or unsubstituted aryl group and a substituted or unsubstituted heteroaryl group;
said aryl group substituents and said heteroaryl group substituents being one or more radical(s) independently selected from the group consisting of alkyl, alkoxy, halogen, phenylamido, a heterocyclic radical, and a substituted or unsubstituted heterocyclosulfonyl;
said heterocyclosulfonyl substituents being one or more radical(s) independently selected from the group consisting of a heteroaryl group; 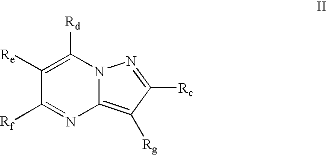
wherein R_{c} represents a radical selected from the group consisting of a substituted or unsubstituted aryl group and —C(═O)NH—Rₕ;
R_{d} represents a radical selected from the group consisting of hydroxy and polyhaloalkyl;
Rₑ represents a radical selected from the group consisting of hydrogen, alkyl, alkenyl, and arylalkyl;
R_{f} represents a radical selected from the group consisting of alkyl, phenyl and a heteroaryl group;
R_{g} represents a radical selected from the group consisting of hydrogen and alkyl;
Rₕ represents a radical selected from the group consisting of cycloalkyl, arylalkyl, and heteroarylalkyl;
said aryl group substituents being one or more radical(s) independently selected from the group consisting of alkyl, alkoxy, and halogen; 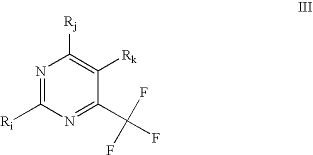
wherein Rᵢ represents a radical selected from the group consisting of amino, hydroxy, and a substituted or unsubstituted heterocyclic radical;
Rⱼ represents a radical selected from the group consisting of a substituted or unsubstituted aryl;
Rₖ represents a radical selected from the group consisting of hydrogen, alkyl, a substituted or unsubstituted aryl, and a substituted or unsubstituted heteroaryl;
said aryl group substituents, said heterocyclic radical substituents, and said heteroaryl group substituents being one or more radical(s) independently selected from the group consisting of alkyl, alkoxy, and halogen; 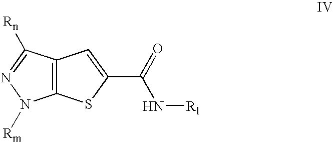
wherein Rₗ and Rₘ represent radicals that are independently selected from the group consisting of a substituted or unsubstituted aryl group and a substituted or unsubstituted heteroaryl group;
and Rₙ represents a radical selected from the group consisting of an alkyl group;
said aryl group substituents and said heteroaryl group substituents being one or more radical(s) independently selected from the group consisting of alkyl, alkoxy, halogen; 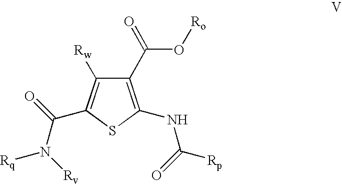
wherein Rₒ represents a radical selected from the group consisting of an alkyl group;
Rₚ represents a radical selected from the group consisting of alkyl, aralkyl, heteroaralkyl, a bicyclic heterocycle, a substituted or unsubstituted aryl group, a substituted or unsubstituted heteroaryl group, and a substituted or unsubstituted aryloxyalkyl group;
R_{q} represents a radical selected from the group consisting of alkyl, cycloalkyl, and a substituted or unsubstituted aryl group;
Rᵥ represents a radical selected from the group consisting of hydrogen and alkyl;
R_{w} represents a radical selected from the group consisting of an alkyl group;
said aryl group substituents, said heteroaryl group substituents, and said aryloxyalkyl group substituents being one or more radical(s) independently selected from the group consisting of alkyl, alkoxy, and halogen; 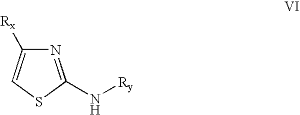
wherein Rₓ represents a radical selected from the group consisting of a substituted or unsubstituted aryl group and a substituted or unsubstituted heteroaryl group;
R_{y} is selected from the group consisting of a substituted or unsubstituted aryl group and a substituted or unsubstituted heteroaryl group;
said aryl group substituents and said heteroaryl group substituents being one or more radical(s) independently selected from the group consisting of alkyl, alkoxy, halogen, carboxyl, amino, amido, alkylcarbonyl, alkoxycarbonyl, and —SO₂—(NH)—R_{z}; and
R_{z} represents a radical selected from the group consisting of hydrogen and a heteroaryl group; 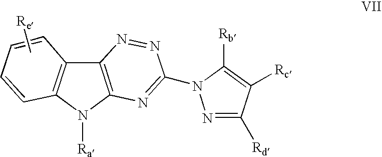
wherein:
R_{a′} represents a radical selected from the group consisting of hydrogen, alkyl, hydroxyalkyl, alkoxyalkyl, alkylthioalkyl, and dialkylaminoalkyl;
R_{b′} represents a radical selected from the group consisting of hydrogen, alkyl, alkoxy, hydroxyalkyl, aryl, and heteroaryl;
R_{c′} represents a radical selected from the group consisting of hydrogen, alkyl, alkoxy, hydroxyalkyl, aryl, and heteroaryl;
R_{d′} represents a radical selected from the group consisting of hydrogen, alkyl, alkoxy, hydroxyalkyl, aryl, and heteroaryl; and
R_{e′} represents a radical selected from the group consisting of alkyl, alkoxy, halogen, monoalkylamino, dialkylamino, and heteroaryl; 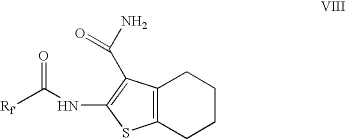
wherein
R_{f+} is selected from the group consisting of alkoxy, benzyl, and a substituted or unsubstituted phenyl;
said phenyl group substituents being one or more radical(s) independently selected from the group consisting of alkyl, alkoxy, and halogen;
and a pharmaceutically acceptable carrier medium.

2. A composition according to claim 1 further comprising at least one supplemental agent selected from the group of interferon, pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

3. A method for prophylaxis or treatment of hepatitis C infections and diseases associated with such infections in a living host having said infections, said method comprising administering to said living host a therapeutically effective amount of at least one compound having the formulas: 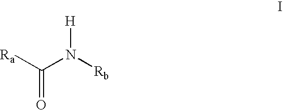
wherein:
Rₐ represents a radical selected from the group consisting of cycloalkyl, a heterocyclic radical, a substituted or unsubstituted aryl group, and a substituted or unsubstituted heteroaryl group;
R_{b} represents a radical selected from the group consisting of a substituted or unsubstituted aryl group and a substituted or unsubstituted heteroaryl group;
said aryl group substituents and said heteroaryl group substituents being one or more radical(s) independently selected from the group consisting of alkyl, alkoxy, halogen, phenylamido, a heterocyclic radical, and a substituted or unsubstituted heterocyclosulfonyl;
said heterocyclosulfonyl substituents being one or more radical(s) independently selected from the group consisting of a heteroaryl group;
and pharmaceutical salts thereof; 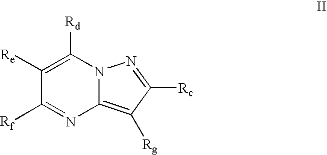
wherein R_{c} represents a radical selected from the group consisting of a substituted or unsubstituted aryl group and —C(═O)NH—Rₕ;
R_{d} represents a radical selected from the group consisting of hydroxy and polyhaloalkyl;
Rₑ represents a radical selected from the group consisting of hydrogen, alkyl, alkenyl, and arylalkyl;
R_{f} represents a radical selected from the group consisting of alkyl, phenyl and a heteroaryl group;
R_{g} represents a radical selected from the group consisting of hydrogen and alkyl;
Rₕ represents a radical selected from the group consisting of cycloalkyl, arylalkyl, and heteroarylalkyl;
said aryl group substituents being one or more radical(s) independently selected from the group consisting of alkyl, alkoxy, and halogen;
and pharmaceutical salts thereof; 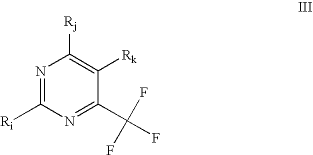
wherein Rᵢ represents a radical selected from the group consisting of amino, hydroxy, and a substituted or unsubstituted heterocyclic radical;
Rⱼ represents a radical selected from the group consisting of a substituted or unsubstituted aryl;
Rₖ represents a radical selected from the group consisting of hydrogen, alkyl, a substituted or unsubstituted aryl, and a substituted or unsubstituted heteroaryl;
said aryl group substituents, said heterocyclic radical substituents, and said heteroaryl group substituents being one or more radical(s) independently selected from the group consisting of alkyl, alkoxy, and halogen;
and pharmaceutical salts thereof; 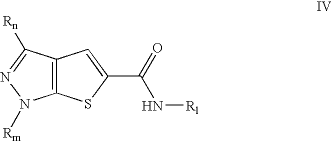
wherein Rₗ and Rₘ represent radicals that are independently selected from the group consisting of a substituted or unsubstituted aryl group and a substituted or unsubstituted heteroaryl group;
and Rₙ represents a radical selected from the group consisting of an alkyl group;
said aryl group substituents and said heteroaryl group substituents being one or more radical(s) independently selected from the group consisting of alkyl, alkoxy, halogen;
and pharmaceutical salts thereof; 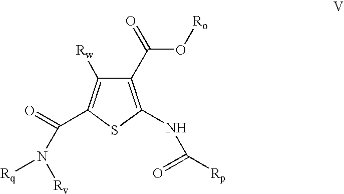
wherein Rₒ represents a radical selected from the group consisting of an alkyl group;
Rₚ represents a radical selected from the group consisting of alkyl, aralkyl, heteroaralkyl, a bicyclic heterocycle, a substituted or unsubstituted aryl group, a substituted or unsubstituted heteroaryl group, and a substituted or unsubstituted aryloxyalkyl group;
R_{q} represents a radical selected from the group consisting of alkyl, cycloalkyl, and a substituted or unsubstituted aryl group;
Rᵥ represents a radical selected from the group consisting of hydrogen and alkyl;
R_{w} represents a radical selected from the group consisting of an alkyl group;
said aryl group substituents, said heteroaryl group substituents, and said aryloxyalkyl group substituents being one or more radical(s) independently selected from the group consisting of alkyl, alkoxy, and halogen;
and pharmaceutical salts thereof; 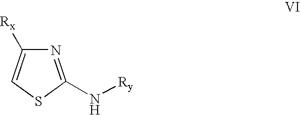
wherein Rₓ represents a radical selected from the group consisting of a substituted or unsubstituted aryl group and a substituted or unsubstituted heteroaryl group;
R_{y} is selected from the group consisting of a substituted or unsubstituted aryl group and a substituted or unsubstituted heteroaryl group;
said aryl group substituents and said heteroaryl group substituents being one or more radical(s) independently selected from the group consisting of alkyl, alkoxy, halogen, carboxyl, amino, amido, alkylcarbonyl, alkoxycarbonyl, and —SO₂—(NH)—R_{z}; and
R_{z} represents a radical selected from the group consisting of hydrogen and a heteroaryl group;
and pharmaceutical salts thereof; 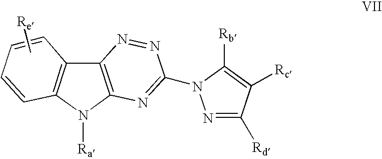
wherein:
R_{a′} represents a radical selected from the group consisting of hydrogen, alkyl, hydroxyalkyl, alkoxyalkyl, alkylthioalkyl, and dialkylaminoalkyl;
R_{b′} represents a radical selected from the group consisting of hydrogen, alkyl, alkoxy, hydroxyalkyl, aryl, and heteroaryl;
R_{c′} represents a radical selected from the group consisting of hydrogen, alkyl, alkoxy, hydroxyalkyl, aryl, and heteroaryl;
R_{d′} represents a radical selected from the group consisting of hydrogen, alkyl, alkoxy, hydroxyalkyl, aryl, and heteroaryl; and
R_{e′} represents a radical selected from the group consisting of alkyl, alkoxy, halogen, monoalkylamino, dialkylamino, and heteroaryl;
and pharmaceutical salts thereof; and 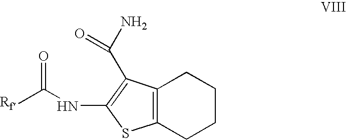
wherein
R_{f′} is selected from the group consisting of alkoxy, benzyl, and a substituted or unsubstituted phenyl;
said phenyl group substituents being one or more radical(s) independently selected from the group consisting of alkyl, alkoxy, and halogen;
and pharmaceutical salts thereof.

4. The method according to claim 3, wherein said living host is a mammal.

5. The method according to claim 3, wherein said living host is a human.

6. The method according to claim 3, wherein the compound is administered orally.

7. The method according to claim 3, wherein the compound is administered orally at a dose range of about 0.05 to about 100 mg/kg.

8. The method according to claim 3, wherein the compound is administered from 1 to 4 times daily.

9. The method according to claim 3, wherein the compound is administered in combination, either concurrently or sequentially, with at least one other biologically active agent.

10. The method according to claim 9, wherein said other biologically active agent is selected from the group consisting of interferon, pegylated interferon, ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

11. A method for preventing apoptosis in a target cell comprising administration of an effective amount of NS4B to said cell.

12. A method for identifying compounds which modulate NS4B signal transduction comprising:
a) providing cells comprising an HCV replicon which express NS4B and exhibit reduced apoptosis;
b) culturing the cells of step a) in the presence and absence of a test compound; and
c) determining whether said compound alters NS4B associated apoptosis relative to untreated cells, thereby identifying a compound effective to inhibit NS4B signal transduction.

13. The method of claim 12, wherein NS4B signal transduction modulates a pathway selected from the group consisting of the interferon signaling pathway, the endoplasmic reticulum stress response pathway, the RNase L pathway, the 2′5′ oligoadenylate pathway and the NFκb pathway.

14. The method of claim 12, wherein said compound inhibits NS4B signal transduction.

15. The method of claim 12, wherein said compound stimulates NS4B signal transduction.

16. A method for identifying compounds which modulate NS4B-associated apoptotic inhibitory activity, comprising:
a) providing a host cell wherein NS4B is expressed;
b) contacting said host cell with a test compound suspected of modulating NS4B associated apoptotic activity;
c) assessing said modulation as a function of alterations in apoptosis levels in the presence of said agent.

17. A kit for practicing the method of claim 16, comprising host cells expressing NS4B, means for determining apoptosis of said cells, and instructional material.

18. A method for identifying compounds having binding affinity for NS4B comprising:
a) providing NS4B protein which is naturally fluorescent;
b) contacting said NS4B protein with an agent suspected of having binding affinity for said NS4B;
c) determining the fluorescence level of said NS4B in the presence and absence of said test compound, those agents which diminish the natural fluorescence of NS4B having binding affinity for NS4B.

19. A compound having NS4B signal transducing inhibitory activity, said compound being effective to induce apoptosis in NS4B expressing cells which exhibit reduced apoptosis in the absence of said compound, said activity being determined by an NS4B binding assay method comprising said compound in the presence of NS4B and determining the binding constant for said compound.

20. A compound according to claim 19, wherein the NS4B is an HCV protein.

21. A method for identifying compounds which modulate HCV NS4B signal transduction comprising:
(a) testing a compound in HCV replicon assay and
(b) testing said compound in HCV protein binding assays

22. A method of distinguishing NS4B biological activity from cellular chemical cytotoxicity for a test compound comprising: (a) measuring the apparent cytotoxicity of a compound in a host cell system, (b) measuring chemical cytotoxicity said compound in said host cell system containing NS4B protein, (c) comparing the results, and (d) identifying the apparent cytotoxicity as corresponding to NS4B biological activity or chemical cytotoxicity.

23. A method of treating an HCV infection in a patient in need of such treatment, said method comprising administering to said patient a pharmaceutically acceptable amount of compound that interacts with NS4B.

24. A method according to claim 23 wherein the said treatment results in modulation of cellular apoptosis.

25. A method according to claim 23 wherein said compound has HCV replicon activity.

26. A method according to claim 23 wherein said compound is identified using an HCV replicon assay and an NS4B binding assay.

27. A method according to claim 23, wherein said compound is administered with at least one agent selected from the group consisting of interferon, a pegylated interferon, ribavirin, a hepatoprotectant, acyclovir, famiclycolvir, valgancyclovir, and amantadine.

28. A compound having NS4B signal transducing inhibitory activity, said compound being effective to induce apoptosis in NS4B expressing cells which exhibit reduced apoptosis in the absence of said compound, said activity being determined by an assay method comprising contacting cells comprising an HCV replicon with said compound and analyzing said cells for apoptosis, said compound inhibiting NS4B signal transduction thereby stimulating apoptosis relative to cells not contacted with said compound.

29. The compound of claim 28, wherein said apoptosis is assessed via a method selected from the group consisting of measurement of DNA integrity, TUNEL assay, and trypan blue exclusion assay.

30. A compound having NS4B inhibitory activity but not having NS4B signal transducing inhibitory activity, said NS4B inhibitory activity being determined by an assay method comprising contacting cells comprising an HCV replicon with said compound, measuring viral replication, and analyzing said compound for NS4B binding activity.

31. A method for inducing apoptosis of hepatitis C-infected cells in a patient, said method comprising administering to said patient a compound of formula VII of claim 1 in an amount effective for induction of apoptosis of hepatitis C-infected cells in said patient, the induction of apoptosis being effected without producing toxemia in said patient.

32. The method of claim 31, wherein said compound is administered during the acute or silent phase of said infection.


SEQUENCE LISTING (4 sequences)

SEQ ID NO: 1 - DNA, 783 bp, Hepatitis C Virus
gcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgc

SEQ ID NO: 2 - protein, 261 aa, Hepatitis C Virus
ASHLPYIEQGMQLAEQFKQKAIGLLQTATKQAEAAAPVVESKWRTLEAFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPC

SEQ ID NO: 3 - DNA, 783 bp, Hepatitis C Virus
  misc_feature (609)...(609): k = g or t
gcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaggcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtagtcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggtcaacctactccctgctatcctctcccctggcgccctagtcgtcggggtcgtgtgcgcagcgatactgcgtcggcacgtgggcccaggggagggggctgtgcaktggatgaaccggctgatatcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgc

SEQ ID NO: 4 - protein, 261 aa, Hepatitis C Virus
  UNSURE (203)...(203): Xaa = any amino acid
ASHLPYIEQGMQLAEQFKQKAIGLLQTATKQAEAAAPVVESKWRTLEAFWARHMWNFISGIQYLAGLSTLPGNPAIASLMAFTASITSPLTTQHTLLFNILGGWVAAQLAPPSAASAFVVAGIAGAAVGSIGLGKVLVDILAGYGAGVAGALVAFKVMSGEMPSTEDLVNLLPAILSPGALVVGVVCAAILRRHVGPGEGAVXWMNRLISFASRGNHVSPTHYVPESDAAARVTQILSSLTITQLLKRLHQWINEDCSTPC